(19) United States
(12) Patent Application Publication
(10) Pub. No.: US 2021/0383918 A1
(43) Pub. Date: Dec. 9, 2021

(54) SYSTEMS AND METHODS FOR PROVISIONING EMERGENCY PROFILES

(71) Applicant: RapidSOS, Inc., New York, NY (US)

(72) Inventors: Michael John MARTIN, Long Island City, NY (US); Riccardo PELLEGRINI, New York, NY (US)

(21) Appl. No.: 17/343,104

(22) Filed: Jun. 9, 2021

Related U.S. Application Data
(60) Provisional application No. 63/036,988, filed on Jun. 9, 2020.

(51) Int. Cl. G16H 40/20; H04L 29/08; G06Q 50/26; G16H 10/60; G16H 40/67; G06F 16/2458

(52) U.S. Cl. CPC G16H 40/20; H04L 67/306; G06F 16/2471; G16H 10/60; G16H 40/67; G06Q 50/26

(57) ABSTRACT

Described herein are systems, devices, methods, and media for generating and displaying emergency profiles. Also provided are systems, methods, and media for providing emergency profiles to emergency service providers for efficient emergency response.

### CROSS-REFERENCE

This application claims the benefit of U.S. Provisional Application No. 63/036,988, filed Jun. 9, 2020, all of which are incorporated herein in their entirety by reference.

### BACKGROUND OF THE INVENTION

A person in an emergency situation may request help using a mobile communication device such as a cell phone to dial a designated emergency number like 9-1-1 or a direct access phone number for the local emergency service provider (e.g. an emergency dispatch center). Relevant emergency information is then communicated to the dispatcher over the phone call. This call is assigned to one or more first responders by the emergency service provider. However, these communications are typically limited to audio calls with narrow functionality since most emergency service providers that receive emergency calls currently lack the capacity for more sophisticated communications. This approach can result in an information gap such as when the caller is unable to verbalize important information relevant to the emergency.

### SUMMARY

One advantage provided by the systems, servers, devices, methods, and media of the instant application is the ability to gather and deliver emergency information that may be pertinent to emergency situations to emergency service providers (ESPs; e.g., public safety answering points, fire departments, police departments, paramedics, police officers, etc.). In some embodiments, an emergency management system (EMS) includes a clearinghouse (also referred to as an “Emergency Clearinghouse”) that functions to obtain or receive emergency data such as enhanced locations (e.g., device-based hybrid location, verified location) and additional data (e.g., medical history, video feeds, sensor data) from various sources (e.g., medical databases, mobile devices of public or first responders, public cameras, police systems, media outlets) and at various times before, during, or after emergency situations and distribute enhanced locations and additional data to ESPs to aid emergency responders in responding to live emergency situations. In some embodiments, the enhanced locations and additional data are delivered by the EMS to a public safety answering point (PSAP). In some embodiments, the enhanced locations and additional data are displayed within a preexisting pathway, such as an Automatic Location Identification (ALI) display. In some embodiments, the enhanced locations and other emergency data are obtained through alternative pathways directly from the devices in the emergency. In some embodiments, relevant emergency data from different sources are consolidated and displayed for rapid and efficient response. In some embodiments, the enhanced locations and emergency data are displayed through a graphical user interface provided by an emergency response application separate from the preexisting ESP system (e.g., PSAP system).

Disclosed herein is a system for emergency communications, the system operative to: collect emergency data comprising identify information for a specific user from two or more sources; generate an emergency profile comprising a plurality of data fields populated with the emergency data for the specific user; store the user profile on a database comprising a plurality of user profiles for a plurality of users; receive an emergency alert regarding the specific user; query the database to locate the emergency profile comprising the plurality of data fields populated with the emergency data for the specific user obtained from two or more sources; and transmit at least a portion of the emergency profile of the specific user to an appropriate emergency service provider (ESP). In some embodiments, a portion of the plurality of data fields is displayed on an ESP device, wherein at least one of the two or more sources of the emergency data is indicated. In some embodiments, the system is operative to collect emergency data by providing a portal for entering emergency data regarding the specific user. In some embodiments, at least a portion of the plurality of data fields for one or more emergency profiles is populated by references to two or more databases. In some embodiments, the two or more sources are queried via a federated query. In some embodiments, retrieved data from the federated query is used to populate the plurality of data fields to generate the emergency profile of the specific user. In some embodiments, the retrieved data from the federated query for a data field is consolidated based on one or more rules for federated query. In some embodiments, the one or more rules comprise at least one of: (i) select data that is recent; (ii) select data from a source with higher reliability; or (iii) select data based on cross-validation from two or more sources. the two or more sources for the federated query is selected based on one or more of location of emergency, type of emergency, identification information, user permissions, ESP authorization and preferences, reliability of the sources. In some embodiments, the emergency profile comprises symptoms, test results, pre-existing conditions, allergies, medications, prescriptions, physician information, medical insurance, in-network medical facilities, emergency contact information, blood type, mental health conditions, substance abuse disorder, developmental disorders, or any combination thereof. In some embodiments, the system is operative to flag the emergency profile of the specific user based on an indication of a mental health condition and provide the ESP with additional options to dispatch mental health professionals in the vicinity of the emergency. In some embodiments, the system is operative to notify one or more emergency contacts in the emergency profile of the emergency.

In some aspects, disclosed herein is a method for providing an emergency profile for emergency response, comprising: collecting emergency data comprising identifying information for a specific user from two or more sources; generating an emergency profile comprising a plurality of data fields populated with the emergency data for the specific user; storing the user profile on a database comprising a plurality of emergency profiles for a plurality of users; receiving an emergency alert regarding the specific user; querying the database to locate the emergency profile comprising the plurality of data fields populated with the emergency data for the specific user obtained from two or more sources; and transmitting at least a portion of the emergency profile of the specific user to an appropriate emergency service provider (ESP). In some embodiments, at least a portion of the plurality of data fields is provided to an ESP device for display, wherein at least one of the two or more sources of the emergency data is indicated. In some embodiments, collecting emergency data comprises providing a portal for entering emergency data regarding the specific user. In some embodiments, at least a portion of the plurality of data fields for one or more emergency profiles is populated by references to two or more databases. In some embodiments, the emergency data comprises retrieved data obtained from the two or more sources using a federated query. In some embodiments, the retrieved data from the federated query is used to populate the plurality of data fields to generate the emergency profile of the specific user. In some embodiments, the retrieved data from the federated query for a data field is consolidated based on one or more rules for federated query. In some embodiments, the one or more rules comprise at least one of: select data that is recent; select data from a source with higher reliability; or select data based on cross-validation from two or more sources. In some embodiments, the two or more sources for the federated query are selected based on one or more of location of emergency, type of emergency, identification information, user permissions, ESP authorization and preferences, reliability of the two or more sources. In some embodiments, the emergency profile comprises symptoms, test results, pre-existing conditions, allergies, medications, prescriptions, vaccination status, physician information, medical insurance, in-network medical facilities, emergency contact information, blood type, mental health conditions, substance abuse disorder, developmental disorders, or any combination thereof. In some embodiments, collecting the emergency data for the specific user comprises providing an online portal allowing the specific user to enter at least a portion of the emergency data and optionally upload one or more files, photos, or videos. In some embodiments, the method further comprises flagging the emergency profile of the specific user based on an indication of a mental health condition and providing the ESP with additional options to dispatch mental health professionals in the vicinity of the emergency. In some embodiments, the method further comprises notifying one or more emergency contacts in the emergency profile of the emergency.

In some aspects, disclosed herein is a system comprising: a) a database comprising a plurality of emergency profiles for a plurality of users; and b) at least one server providing an emergency management system server application, wherein the application is operative to: collect emergency data comprising identifying information for a specific user from two or more sources; generate an emergency profile comprising a plurality of data fields populated with the emergency data for the specific user; store the user profile on the database comprising the plurality of emergency profiles for the plurality of users; receive an emergency alert regarding the specific user; query the database to locate the emergency profile comprising the plurality of data fields populated with the emergency data for the specific user obtained from two or more sources; and transmit at least a portion of the emergency profile of the specific user to an appropriate emergency service provider (ESP). In some embodiments, the application is operative to provide at least a portion of the plurality of data fields to an ESP device for display, wherein at least one of the two or more sources of the emergency data is indicated. In some embodiments, the system is operative to provide a portal for entering emergency data regarding the specific user. In some embodiments, at least a portion of the plurality of data fields for one or more emergency profiles is populated by references to two or more databases. In some embodiments, the emergency data comprises retrieved data obtained from the two or more sources using a federated query. In some embodiments, the retrieved data from the federated query is used to populate the plurality of data fields to generate the emergency profile of the specific user. In some embodiments, the retrieved data from the federated query for a data field is consolidated based on one or more rules for federated query. In some embodiments, the one or more rules comprise at least one of: select data that is recent; select data from a source with higher reliability; or select data based on cross-validation from two or more sources. In some embodiments, the two or more sources for the federated query are selected based on one or more of location of emergency, type of emergency, identification information, user permissions, ESP authorization and preferences, reliability of the two or more sources. In some embodiments, the emergency profile comprises symptoms, test results, pre-existing conditions, allergies, medications, prescriptions, vaccination status, physician information, medical insurance, in-network medical facilities, emergency contact information, blood type, mental health conditions, substance abuse disorder, developmental disorders, or any combination thereof. In some embodiments, the system is operative to provide an online portal allowing the specific user to enter at least a portion of the emergency data and optionally upload one or more files, photos, or videos. In some embodiments, the system is operative to flag the emergency profile of the specific user based on an indication of a mental health condition and provide the ESP with additional options to dispatch mental health professionals in the vicinity of the emergency. In some embodiments, the system is operative to notify one or more emergency contacts in the emergency profile of the emergency.

Disclosed herein is a system for emergency communications, comprising: (a) a portal configured to provide emergency profiles for a plurality of users; and (b) at least one server providing an emergency management system server application, wherein the application is operative to: (i) maintain a database comprising the emergency profiles for the plurality of users, each of the emergency profiles comprising a plurality of data fields for emergency data; (ii) receive an emergency alert regarding a specific user; (iii) query the database to locate an emergency profile of the specific user within the database; and (iv) transmit at least a portion of the emergency profile of the specific user to an appropriate emergency service provider (ESP). In some embodiments, the emergency profile comprises symptoms, test results, pre-existing conditions, allergies, medications, prescriptions, physician information, medical insurance, in-network medical facilities, emergency contact information, or any combination thereof. In some embodiments, the emergency profile comprises mental health conditions, substance abuse disorders, developmental disorders, or any combination thereof. In some embodiments, the appropriate ESP is a behavioral health expert. In some embodiments, the emergency profile comprises Covid-19 symptoms, Covid-19 test results, Covid-19 diagnosis, vaccination status, or any combination thereof. In some embodiments, the emergency profile comprises one or more emergency contacts. In some embodiments, the emergency contacts are authorized to receive information about the specific user based on input or instructions from the specific user. In some embodiments, emergency contacts are authorized to receive information about the specific user during an emergency. In some embodiments, the emergency contacts comprise a primary physician of the specific user. In some embodiments, the application is further operative to inform at least one of the emergency contacts of the emergency alert regarding the specific user. In some embodiments, the application is further operative to add at least one of the emergency contacts to an on-going emergency call or emergency communication session. In some embodiments, the application is further operative to update the emergency profile of the specific user with recent or current emergency data. In some embodiments, the emergency profile of the specific user comprises emergency data entered by the specific user. In some embodiments, the emergency profile of the specific user comprises emergency data entered by another user associated with the specific user. In some embodiments, another user is an emergency contact of the specific user. In some embodiments, the emergency profile of the specific user comprises emergency data from one or more devices associated with the specific user. In some embodiments, the emergency profile of the specific user comprises emergency data from one or more third-party servers. In some embodiments, the emergency profile of the specific user comprises emergency data from two or more third-party servers. In some embodiments, the application is further operative to send queries about the specific user to two or more third-party servers.

In some embodiments, the system is further configured to send a federated query to one or more third-party databases in addition to the database of Emergency Profiles (EP). In some embodiments, the federated query is searched using a name, a phone number, an email address, an IP address SSN or another unique identifier of the specific user. In some embodiments, the federated query is searched using a first identifier of the specific user in one database and a second identifier in a second database. In some embodiments, the retrieved data from the federated query is displayed at the ESP in a form optimized for emergency response. In some embodiments, the retrieved data from the federated query is displayed at a PSAP in standardized form. In some embodiments, the retrieved data from the federated query is displayed based on factors such as emergency type, priority, relevance, privacy controls, user permissions, ESP credentials. In some embodiments, the retrieved data from the federated query is displayed at an ESP indicating one or more sources of the data. In some embodiments, the retrieved data from the federated query is displayed at an ESP based on one or more rules of relevance. In some embodiments, the retrieved data from the federated query is consolidated to remove discrepancies and errors. In some embodiments, the retrieved data from the federated query is consolidated by cross-validating between two sources. In some embodiments, the retrieved data from the federated query is consolidated to remove stale data (e.g., non-current data such as location data exceeding a time threshold). In some embodiments, the collection of the emergency healthy profiles is saved in an emergency profile database. In some embodiments, the emergency profile comprises emergency data and are stored and transmitted in encrypted form. In some embodiments, the system further comprises an encryption tool for encrypting information in the emergency profiles. In some embodiments, the system further comprises a decryption tool for decrypting the encrypted information in the emergency profiles. In some embodiment, location-based encryption and decryption is used to encrypt information in the emergency profiles. In some embodiments, the system is further configured to remove identifiable information about the specific user from the emergency profile to create an anonymized emergency profile. In some embodiments, the anonymized emergency profile is transmitted to public health entities and/or medical providers. In some embodiments, the emergency profile of the specific user is queried using a first and last name, phone number, email address, address, or any combination thereof.

Disclosed herein is a method for facilitating emergency communications, comprising: providing a portal configured to provide emergency profiles for a plurality of users;

maintaining a database comprising the emergency profiles for the plurality of users, each of the emergency profiles comprising a plurality of data fields for emergency data; receiving an emergency alert regarding a specific user; querying the database to locate an emergency profile of the specific user within the database; and transmitting at least a portion of the emergency profile of the specific user to an appropriate emergency service provider (ESP). In some embodiments, the emergency profile comprises symptoms, test results, pre-existing conditions, allergies, medications, prescriptions, physician information, medical insurance, in-network medical facilities, emergency contact information, or any combination thereof. In some embodiments, the emergency profile comprises Covid-19 symptoms, Covid-19 test results, Covid-19 diagnosis, or any combination thereof. In some embodiments, the emergency profile comprises one or more emergency contacts. In some embodiments, the emergency contacts are authorized to receive information about the specific user based on input or instructions from the specific user. In some embodiments, the emergency contacts are authorized to receive information about the specific user during an emergency. In some embodiments, the emergency contacts comprise a primary physician of the specific user. In some embodiments, the application is further operative to inform at least one of the emergency contacts of the emergency alert regarding the specific user. In some embodiments, the application is further operative to add at least one of the emergency contacts to an on-going emergency call or emergency communication session. In some embodiment, the application is further operative to update the emergency profile of the specific user with recent or current emergency data. In some embodiments, the emergency profile of the specific user comprises emergency data entered by the specific user. In some embodiments the emergency profile of the specific user comprises emergency data entered by another user associated with the specific user. In some embodiments, another user is an emergency contact of the specific user. In some embodiments, the emergency profile of the specific user comprises emergency data from one or more third-party servers. In some embodiments, the emergency profile of the specific user comprises emergency data from two or more third-party servers. In some embodiments, the application is further operative to send queries about the specific user to two or more third-party servers.

In some embodiments, the method is further configured to send a federated query to one or more third-party databases. In some embodiments, the federated query is searched using a name, a phone number, an email address, an IP address SSN or another unique identifier of the specific user. In some embodiments, the federated query is searched using a first identifier of the specific user in one database and a second identifier in a second database. In some embodiments, the retrieved data from the federated query is displayed at the ESP in a form optimized for emergency response. In some embodiments, the retrieved data from the federated query is displayed at a PSAP in standardized form. In some embodiments, the retrieved data from the federated query is displayed based on factors such as emergency type, priority, relevance, privacy controls, user permissions, ESP credentials. In some embodiments, the retrieved data from the federated query is displayed at an ESP indicating one or more sources of the data. In some embodiments, the retrieved data from the federated query is displayed at an ESP based on one or more rules of relevance. In some embodiments, the retrieved data from the federated query is consolidated to remove discrepancies and errors. In some embodiments, the retrieved data from the federated query is consolidated by cross-validating between two sources. In some embodiments, the retrieved data from the federated query is consolidated to remove stale data. In some embodiments, the collection of the emergency healthy profiles is saved in an encrypted emergency profile database. In some embodiments, the method further comprises a decryption tool for decrypting the encrypted information in the emergency profiles. In some embodiments, the location-based encryption and decryption is used to encrypt information in the emergency profiles. In some embodiments, the method is further configured to remove identifiable information about the specific user from the emergency profile to create an anonymized emergency profile. In some embodiments, the anonymized emergency profile is transmitted to one or more of public health entities, medical providers, behavioral health specialists.

In some aspects, disclosed herein is non-transitory computer readable storage medium comprising instructions executable by a processor to carry out any of the described methods disclosed herein.

### BRIEF DESCRIPTION OF DRAWINGS

Disclosed herein are systems, devices, The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting embodiment of the emergency response data platform (ERDP);

FIG. 2 depicts a non-limiting embodiment of an emergency flow management system (EFMS);

FIG. 3 depicts a non-limiting embodiment of an interface for the emergency response application depicting a list of emergencies;

FIGS. 4A & 4B depict a non-limiting embodiment of flows for sending emergency alerts to an emergency service provider (ESP);

FIG. 5 depicts a non-limiting embodiment of components of the (i) ERDP and (ii) ESP system;

FIG. 6 depicts a non-limiting embodiment of a method for generating and sharing an emergency profile (EP);

FIGS. 7A & 7B depict a non-limiting embodiment of interfaces for providing a portal for users to register for an EP;

FIGS. 8A, 8B, 8C, & 8D illustrate a non-limiting embodiment of an interface for a user to provide information for an EP;

FIGS. 9A & 9B depicts a non-limiting embodiment of a method for implementing federated queries for emergency data; and

FIGS. 10 & 11 depicts non-limiting embodiments of interfaces displayed at an ESP system.

### DETAILED DESCRIPTION

Disclosed herein are systems, devices, media, and methods for providing enhanced emergency communications and functions. Embodiments of the present disclosure take advantage of technological advancements that have allowed for mobile communication devices to generate accurate locations by incorporating multiple technologies embedded in the devices, such as GPS, Wi-Fi, and Bluetooth to create device-based hybrid locations. Many of these devices are Internet-abled and can communicate via the Internet by sending messages such as chat, email, HTTP post, etc.

### Emergency Response Data Platform

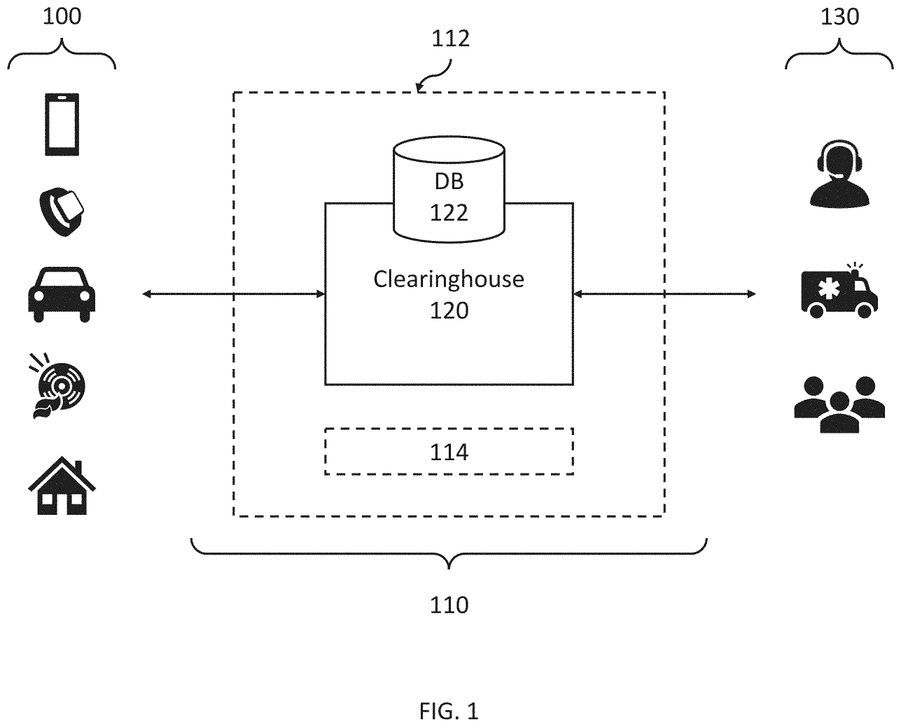

In various embodiments, disclosed herein are devices, systems, and methods for managing emergency data and emergency communications for more effective and efficient emergency response. FIG. 1 depicts a diagram of an emergency response data platform in accordance with one embodiment of the present disclosure. In a simple example, in some embodiments, an emergency data source 100 transmits emergency to an emergency response data platform (ERDP) 110 before, during, or after an emergency, and the emergency response data platform shares the emergency data with an emergency service provider (ESP) 130. Non-limiting examples of ingress data sources includes mobile phones, wearables, vehicle telematics systems, smart security systems, and mobile applications. The ESP 130 can then use the emergency data to more efficiently and effectively respond to corresponding emergencies. In some embodiments, the emergency data source 100 is a third-party server system (hereinafter, “third-party server”). For example, in some embodiments, the emergency data source 100 is a third-party server (e.g., a backend server system) of a technology company that produces software for electronic devices, such as Apple or Google. In some embodiments, the emergency data source 100 is an electronic device, such as an electronic communication device. For example, the emergency data source 100 may be a communication device (e.g., a walkie talkie or two-way radio, a mobile or cellular phone, a computer, a laptop, etc.), a wearable device (e.g., a smartwatch), or an Internet of Things (IoT) device such as a home assistant (e.g., an Amazon Echo) or a connected smoke detector (e.g., a Nest Protect smoke and carbon monoxide alarm). In some embodiments, an electronic device includes a display, a processor, a memory (e.g., an EPROM memory, a RAM, or a solid-state memory), a network component (e.g., an antenna and associated components, Wi-Fi adapters, Bluetooth adapters, etc.), a data storage, a user interface, an emergency alert program, one or more location components, and one or more sensors. In some embodiments, the processor is implemented as one or more microprocessors, microcomputers, microcontrollers, digital signal processors, central processing units, state machines, logic circuitries, and/or devices that manipulate signals based on operational instructions. Among other capabilities, the processor is configured to fetch and execute computer-readable instructions stored in the memory.

In some embodiments, the display is part of the user interface (e.g., a touchscreen is both a display and a user interface in that it provides an interface to receive user input or user interactions). In some embodiments, the user interface includes physical buttons such as an on/off button or volume buttons. In some embodiments, the display and/or the user interface comprises a touchscreen (e.g., a capacitive touchscreen), which is capable of displaying information and receiving user input. In some embodiments, the communication device includes various accessories that allow for additional functionality. In some embodiments, these accessories (not shown) include one or more of the following: a microphone, a camera, speaker, a fingerprint scanner, health or environmental sensors, a USB or micro-USB port, a headphone jack, a card reader, a SIM card slot, or any combination thereof. In some embodiments, the one or more sensors include, but are not limited to: a gyroscope, an accelerometer, a thermometer, a heart rate sensor, a barometer, or a hematology analyzer. In some embodiments, the data storage includes a location data cache and a user data cache. In some embodiments, the location data cache is configured to store locations generated by the one or more location components.

In some embodiments, the emergency alert program is a web application or mobile application. In some embodiments, the emergency alert program is configured to record user data, such as a name, address, or medical data of a user associated with the electronic device. In some embodiments, the emergency alert program is configured to detect when an emergency request is generated or sent by the electronic device (e.g., when a user uses the electronic device to make an emergency call). In some embodiments, in response to detecting an emergency request generated or sent by the electronic device, the emergency alert program is configured to deliver a notification to the ERDP 110. In some embodiments, the notification is an HTTP post containing information regarding the emergency request. In some embodiments, the notification includes a location (e.g., a device-based hybrid location) generated by or for the electronic device. In some embodiments, in response to detecting an emergency request generated or sent by the electronic device, the emergency alert program is configured to deliver user data to the ERDP 110.

In some embodiments, the emergency response data platform (ERDP) 110 includes an ERDP operating system, an ERDP CPU, an ERDP memory unit, an EMS communication element, and one or more software modules. In some embodiments, the ERDP CPU is implemented as one or more microprocessors, microcomputers, microcontrollers, digital signal processors, central processing units, state machines, logic circuitries, and/or devices that manipulate signals based on operational instructions. Among other capabilities, the ERDP CPU is configured to fetch and execute computer-readable instructions stored in the ERDP memory unit. The ERDP memory unit optionally includes any computer-readable medium known in the art including, for example, volatile memory, such as static random-access memory (SRAM) and dynamic random-access memory (DRAM), and/or non-volatile memory, such as read-only memory (ROM), erasable programmable ROM, flash memories, hard disks, optical disks, and magnetic tapes. The ERDP memory unit optionally includes modules, routines, programs, objects, components, data structures, etc., which perform particular tasks or implement particular abstract data types.

In some embodiments, the ERDP 110 includes one or more ERDP databases, one or more servers, and a clearinghouse 120. In some embodiments, the clearinghouse 120, as described in further detail below, is an input/output (I/O) interface configured to manage communications and data transfers to and from the ERDP 110 and external systems and devices. In some embodiments, the clearinghouse 120 includes a variety of software and hardware interfaces, for example, a web interface, a graphical user interface (GUI), and the like. The clearinghouse 120 optionally enables the ERDP 110 to communicate with other computing devices, such as web servers and external data servers. In some embodiments, the clearinghouse 120 facilitates multiple communications within a wide variety of networks and protocol types, including wired networks, for example, LAN, cable, etc., and wireless networks, such as WLAN, cellular, or satellite. In some embodiments, the clearinghouse 120 includes one or more ports for connecting a number of devices to one another or to another server. In some embodiments, the clearinghouse 120 includes one or more sub-clearinghouses, such as location clearinghouse and additional data clearinghouse, configured to manage the transfer of locations and additional data, respectively. In some embodiments, the clearinghouse 120 can be queried using for location data using an API (e.g., LIS App) and additional data using another API (e.g., ADR App).

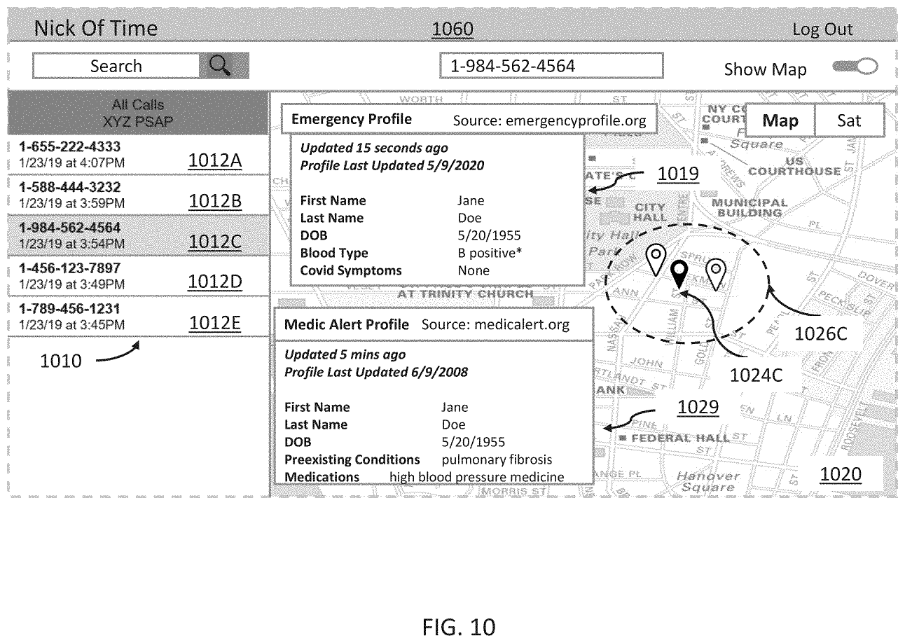

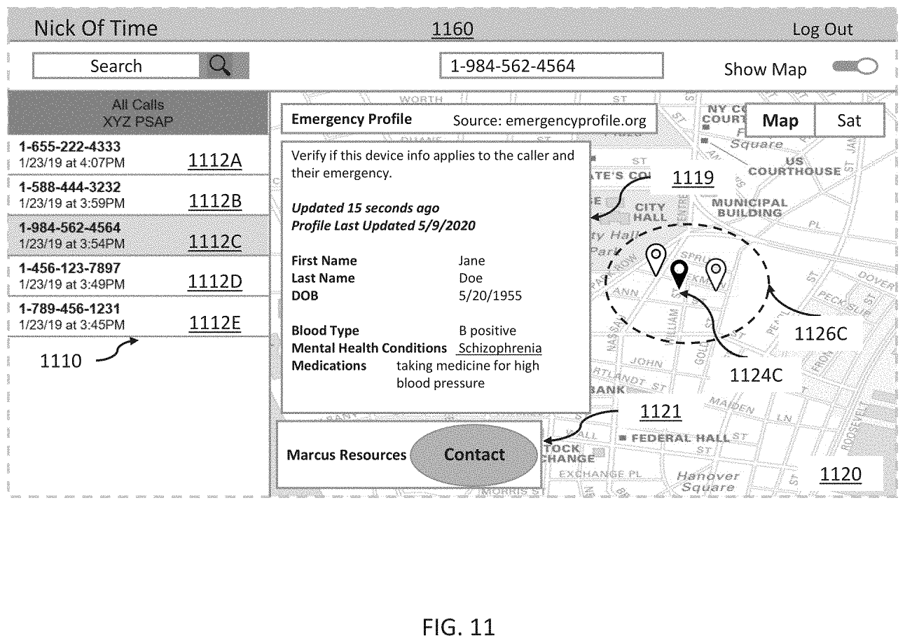

In some embodiments, the clearinghouse 120 can query one or more additional sources for information about the emergency using a user identifier (e.g., a phone number, name, social security, address, IMEI, SIM number, alarm ID, etc.). For example, the clearinghouse 120 may query an emergency profile (EP) database after an emergency alert has been triggered. and the retrieved data may be displayed as shown in FIG. 10. In some embodiments, the clearinghouse 120 can query two or more sources via a federated query.

In other embodiments, the clearinghouse 120 is populated with critical user data (e.g., pre existing conditions, mental health diagnosis, etc.) from the EP database even before an emergency has taken place. In this way, critical user data may be available quicker for routing the emergency service request to an appropriate ESP. Critical user data may be identified by different agencies based on rules and procedures within the jurisdiction. For example, a particular ESP may flag specific diagnoses such as Covid-19, schizophrenia, etc. as critical user data that should be available and can be saved in the clearinghouse 120 before the emergency has been triggered.

In some embodiments, the ERDP 110 additionally includes a user information module that receives and stores user information (e.g., personal information, demographic information, medical information, location information, etc.) within the ERDP 110. In some embodiments, users can submit user information through a website, web application, or mobile application, such as during a registration process for an emergency response application. In some embodiments, when the ERDP 110 receives emergency data including user information, such as through an emergency alert received by the clearinghouse 120 (as described below), the ERDP 110 stores the user information in the user information module. In some embodiments, user information stored within the user information module is received by the ERDP 110 from a third-party server system, as described above. In some embodiments, user information stored within the user information module is associated with an identifier of a user or an electronic device associated with a user, such as a phone number or an email address.

In some embodiments, the location data is generated by the electronic device before the emergency call and can be made accessible by the ERDP to an ESP during an emergency. For example, a taxi company may have software that transmits the location of its cars or assets to the emergency clearinghouse preemptively. Thus, when an emergency call is made, the location of the affected taxi can be made accessible quicker to send help. In addition, the ERDP may transmit emergency alerts even when the emergency call does not go through or gets disconnected based on the emergency data from the electronic device.

Using the ERDP 110 two-sided platform, relevant emergency data about one or more emergencies can be shared with one or more egress recipients, such as a call center (e.g., PSAP), emergency responders, or emergency contacts in a secure and efficient manner. Using the platform, authorized recipients are given access to relevant information for quick and efficient emergency response. For example, a PSAP is enabled to verify the location of an emergency caller via technology, rather than relying on a distressed caller to generate the location data. Thus, a PSAP can initiate a response before the user provides any location information, saving seconds or minutes on emergency response time.

In some cases, a primary agency (e.g., PSAP-1) may select an appropriate secondary agency to respond to the emergency. In some embodiments, a federal agency such as the Center for Disease Control (CDC) is planning and overseeing various state and local ESP agencies for dealing with outbreak of infectious diseases.

In some implementations, the emergency service request, such as an emergency call, may be retained by the primary agency while the secondary agency dispatches emergency responders to the emergency location. In other implementations, the emergency service request, such as an emergency call is transferred to the appropriate secondary agency based on emergency location, type and priority of the emergency.

A primary agency may be responsible for handling emergency service requests (such as traditional emergency calls and digital requests) within an authoritative jurisdiction, which may be defined by one or more geofences. A buffer region may be defined around the boundary of the geofence and the locations falling within the buffer region can be treated as locations falling within the geofence. The buffer region may be 1 meter to 10 km, or between 200 meters to 5 km, preferably 2 km. A secondary agency may also have a geofence, which defines the area of operation, but the service request regarding an emergency has to be initiated by a primary agency.

In addition, other types of ESP agencies are also contemplated. For example, ESPs may be temporarily set up to address a particular threat or a natural disaster, such as a hurricane. The constituting ESP agencies for such a “temporary ESP agency” may be the area that might be impacted by the threat or natural disaster. It is also contemplated that ESP agencies may be both public and private entities such as corporate security, university police, call center, etc.

As mentioned above, in some embodiments, the emergency response data platform (ERDP) 110 shares emergency data with an emergency service provider (ESP) 130. In some embodiments, an ESP 130 (e.g., a public safety answering point (PSAP)) is a system that includes one or more of a display, a user interface, at least one central processing unit or processor, a network component, an audio system, (e.g., microphone, speaker and/or a call-taking headset), and an ESP application (e.g., a computer program) such as a computer aided dispatch (CAD) program or an emergency call taking program (also referred to as customer premise equipment or CPE). In some embodiments, the ESP application comprises a database of emergency responders, such as medical assets, police assets, fire response assets, rescue assets, safety assets, etc. In some embodiments, the ESP application is an emergency response application provided by the ERDP 110, as described below. In some embodiments, the ESP application is installed on a computing device at the ESP 130 and comprise one or more software modules, such as a call taking module, an ESP display module, a supplemental or updated information module, or a combination thereof. In some embodiments, the ESP application displays the information on a map (e.g., on the display). In some embodiments, the ESP application is accessible or executable on mobile devices associated with ESP 130, such as first responder devices. In some embodiments, the ESP application is an emergency response application provided by the ERDP 110, as described below.

### Emergency Clearinghouse

In some embodiments, as mentioned above with respect to FIG. 1, the emergency response data platform (ERDP) 110 includes a clearinghouse 120 (also referred to as an “Emergency Clearinghouse”) for receiving, storing, retrieving, and transmitting emergency data. In some embodiments, as depicted by FIG. 1, through the clearinghouse 120, the ERDP 110 can receive emergency data from an emergency data source 100 (as described above) and transmit the emergency data to an emergency data recipient, such as an emergency service provider (ESP) 130 (as described above). In this way, the ERDP 110 acts as a data pipeline between emergency data sources 100 and ESPs 130. The emergency data that passes through the clearinghouse 120 may include (but is not limited to) location data (e.g., fixed addresses or device-based hybrid locations generated in real time) and additional data (e.g., medical history, personal information, or contact information, etc.). In some embodiments, through the clearinghouse 120, the ERDP 110 transmits emergency data to ESPs 130 to aid the ESPs 130 in responding to emergencies. For example, location data may allow emergency responders to arrive at the scene of an emergency faster, and additional data may allow emergency responders to be better prepared for the emergencies that they face.

The clearinghouse 120 may receive emergency data in various ways. For example, in some embodiments, an emergency data source 100 can unilaterally transmit emergency data to the clearinghouse 120. For example, in one embodiment, an emergency alert is triggered by an electronic device manually (e.g., in response to the selection of a soft or hard emergency button) or automatically based on sensor data received by the electronic device (e.g., smoke alarms). The electronic device can then transmit the emergency alert and any associated data to the ERDP 110, such as to an endpoint provided by the clearinghouse 120. Or, for example, in one embodiment, after an emergency alert is received by the ERDP 110 from a first emergency data source, the ERDP 110 can query a second emergency data source for emergency data (e.g., emergency data associated with the emergency alert received from the first emergency data source). For example, the emergency alert received from the first emergency data source may include a user identifier (e.g., a telephone number or an email address) for an owner or user of the first emergency data source. The ERDP 110 can then query the second emergency data source with the user identifier to retrieve additional emergency data associated with the owner or user of the first emergency data source. In some embodiments, emergency data received by the ERDP 110 is received in a format that is compatible with industry standards for storing and sharing emergency data. In some embodiments, the ERDP 110 formats emergency data that it receives into a format that is compatible with industry standards. For example, in some embodiments, the emergency data is formatted to be compatible with National Emergency Number Association (NENA) standards. In some embodiments, emergency data is formatted by the ERDP 110 to be compliant with the Presence Information Data Format Location Object (PIDF-LO) standard. In some embodiments, emergency data received by the ERDP 110 is stored within one or more databases 122. In some embodiments, emergency data received by the ERDP 110 is associated with one or more identifiers, such as a device or user identifier.

The clearinghouse 120 may share emergency data in various ways. For example, in some embodiments, an emergency data recipient, such as an ESP 130, can query the ERDP 110 for emergency data. For example, in some embodiments, an ESP 130 can query the ERDP 110 with a user identifier (e.g., a telephone number or an email address) to receive emergency data gathered or received by the ERDP 110 associated with the user identifier. Or for example, in some embodiments, an ESP 130 can query the ERDP 110 with a geospatial area to receive emergency data gathered or received by the ERDP 110 associated with the geospatial area. Alternatively, in some embodiments, the ERDP 110 can autonomously transmit emergency data to an emergency data recipient without first receiving a query from the emergency data recipient (also referred to as “pushing” emergency data, as opposed to emergency data being “pulled” with a query). In some embodiments, the ERPD 110 pushes emergency data to an emergency data recipient using an emergency data subscription system. Using the emergency data subscription system, an emergency data recipient can subscribe to the clearinghouse 120 for a particular device identifier, user identifier, ESP account, or geospatial area. After subscribing to a subscription, the emergency data recipient may automatically receive updates regarding the subscription without first sending a query for emergency data. For example, if an ESP 130 subscribes to a phone number, whenever the ERDP 110 receives updated emergency data associated with the phone number, the clearinghouse 120 can instantly and automatically transmit the updated emergency data associated with the phone number to the ESP 130.

As used herein, “emergency data” refers to data pertaining to an on-going or historical emergency. The emergency data may be generated at the time of the emergency. The emergency data may be generated before the emergency occurs and may be made accessible when the emergency occurs. In some embodiments, the emergency data comprises location data, particularly the current location of the emergency (often times based on the location of the user device). Because of privacy and security concerns, emergency data must be stored, accessed, transmitted using security and privacy measures.

In some embodiments, location data comprises a location of a device determined using a location determination method. In further embodiments, a location determination method is selected from GPS satellite triangulation, cell tower triangulation, Wi-Fi triangulation, Bluetooth triangulation, RSSI, time-of-flight, angle of arrival, fingerprinting, barometric pressure, or any combination thereof. In some embodiments, location is determined using more than one method in combination to obtain a more accurate location. In some embodiments, location data comprises coordinates (e.g., XYZ coordinates, longitude, latitude, altitude, etc), an address (e.g., an address equivalent to coordinates that provides a current dispatchable location for emergency response). In some embodiments, location data comprises historical location (e.g., where a user has traveled in the past). In some embodiments, historical location comprises one or more locations of the user and/or user device equal to or greater than 1, 2, 3, 4, 5, 10, 15, 20, 25, 30, 40, 50, or 60 minutes old, including increments therein. In some embodiments, the historical location comprises one or more locations of the user and/or user device equal to or greater than 1, 2, 3, 4, 5, 6, 7, 8, 9, 10, 11, 12, 13, 14, 15, 16, 17, 18, 19, 20, 21, 22, 23, or 24 hours old. In some embodiments, location data comprises current location, wherein current location comprises one or more locations of the user and/or user device within the past 1, 2, 3, 4, 5, 10, 15, 20, 25, 30, 40, 50, or 60 minutes, including increments therein. In some embodiments, location data comprises current location, wherein current location comprises one or more locations of the user and/or user device within the past 1, 2, 3, 4, 5, 6, 7, 8, 9, 10, 11, 12, 13, 14, 15, 16, 17, 18, 19, 20, 21, 22, 23, or 24 hours.

As used herein, “sensor data” refers to information obtained or provided by one or more sensors. In some instances, a sensor is associated with a device (e.g., user has a communication device with a data link via Bluetooth with a wearable sensor, such as, for example, a heart rate monitor or a pedometer). Accordingly, in some embodiments, the device obtains sensor data from the sensor (e.g., heart rate from the heart rate monitor or distance traveled from the pedometer). In some instances, the sensor data is relevant to an emergency situation (e.g., heart rate during a cardiac emergency event). In some embodiments, a sensor and/or sensor device comprises an acoustic sensor, a breathalyzer, a carbon dioxide sensor, a carbon monoxide sensor, an infrared sensor, an oxygen sensor, an ozone monitor, a pH sensor, a smoke detector, a current sensor (e.g., detects electric current in a wire), a magnetometer, a metal detector, a radio direction finder, a voltage detector, an air flow meter, an anemometer, a flow sensor, a gas meter, a water meter, a Geiger counter, an altimeter, an air speed indicator, a depth gauge, a gyroscope, a compass, an odometer, a shock detector (e.g., on a football helmet to measure impact), a barometer, a pressure gauge, a thermometer, a proximity sensor, a motion detector (e.g., in a home security system), an occupancy sensor, or any combination thereof, and in some embodiments, sensor data comprises information obtained from any of the preceding sensors. In some embodiments, one or more sensors are physically separate from a user device. In further embodiments, the one or more sensors authorize the user device to obtain sensor data. In further embodiments, the one or more sensors provide or send sensor data to the user device autonomously. In some embodiments, the user device and the one or more sensors belong to the same group of devices, wherein member devices are authorized to share data. In some embodiments, a user device comprises one or more sensors (e.g., user device is a wearable device having a sensor or sensing component).

In some embodiments, emergency data comprises current data. In further embodiments, current data comprises information that is equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 minutes old, including increments therein. In further embodiments, current data comprises information that equal to or less than 1, 2, 3, 4, 5, 6, 7, 8, 9, 10, 11, 12, 13, 14, 15, 16, 17, 18, 19, 20, 21, 22, 23, or 24 hours old. In some embodiments, data comprises historical data. In further embodiments, historical data comprises information that is equal to or more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 minutes old, including increments therein. In further embodiments, historical data comprises information that equal to or more than 1, 2, 3, 4, 5, 6, 7, 8, 9, 10, 11, 12, 13, 14, 15, 16, 17, 18, 19, 20, 21, 22, 23, or 24 hours old. In some embodiments, the age of information is calculated from the date the information is first collected (e.g., when a sensor first detects a sensed parameter such as, for example, heart rate).

### Emergency Data Geofencing

In some embodiments, a geofence system 112 is applied to the clearinghouse 120 or the ERDP 110 to ensure that emergency data reaches authorized recipients and to protect sensitive emergency data from being shared with unintended recipients. As depicted in FIG. 1, in some embodiments, when emergency data (e.g., an emergency location or additional data) is received by the ERDP 110 from an emergency data source 100, the emergency data is first processed by the geofence system 112 before being ingested by the clearinghouse 120. Similarly, in some embodiments, when a data request for emergency data is received by the ERDP 110 from an emergency data recipient (e.g., an ESP 130), the query is processed by the geofence system 112 before emergency data is transmitted to the emergency data recipient.

Generally, a geofence is a virtual perimeter that represents a real-world geographic area. A geofence can be dynamically generated—as in a radius around a point location—or a geofence can be a predefined set of boundaries (such as school zones or neighborhood boundaries). For emergency response, an emergency service provider (public or private entities) may be given jurisdictional authority to a certain geographical region or jurisdiction (also referred to as “authoritative regions”). In the context of emergency services, one or more geofences may correspond to the authoritative region of an ESP. In many cases, the ESP is a public entity such as a public safety answering point (PSAP) or a public safety service (PSS; e.g., a police department, a fire department, a federal disaster management agency, national highway police, etc.), which have jurisdiction over a designated area (sometimes, overlapping areas). Geofences are used to define the jurisdictional authority by various methods and in various Geographic Information System (GIS) formats. In some embodiments, geofences only represent authoritative regions if the geofence has been assigned or verified by a local, state, or federal government. In some embodiments, geofences represent assigned jurisdictions that are not necessarily authoritative regions. For example, in some embodiments, a geofence is unilaterally created by its associated ESP without verification or assignment by a local, state, or federal government.

In some embodiments, the ERDP 110 maintains a geofence database including one or more geofences associated with each ESP 130 that is or has ever been communicatively coupled to the ERDP 110. In some embodiments, a geofence associated with an ESP 130 may be submitted to the ERDP 110 by an administrator of the ESP 130, such as through an emergency response application (as described below) or via email. In some embodiments, when emergency data is received by the ERDP 110 the ERDP 110 identifies a location associated with the emergency data (e.g., an emergency location included in an emergency alert) and determines if the location is within the combined authoritative jurisdiction (i.e., within any one of the geofences stored in the geofence database). In some embodiments, if the location is not within the combined authoritative jurisdiction, the ERDP 110 rejects or drops the emergency data (also referred to as “ingress filtering”). In some embodiments, when the ERDP 110 receives a query for emergency data from an ESP 130, the ERDP 110 identifies a geofence associated with the ESP 130 and returns only emergency data associated with locations that are within the geofence associated with the ESP 130 (also referred to as “egress filtering). In some embodiments, geofences are used in routing emergency data that is pushed to an emergency data recipient. In some embodiments, example, as mentioned above, an emergency data recipient may subscribe to an ESP jurisdiction, or specifically to a geofence associated with the ESP (often times the authoritative jurisdiction of the ESP). Then, when the ERDP 110 receives emergency data associated with a location that is within the geofence to which the emergency data recipient has subscribed, the ERDP 110 can instantly and automatically push the emergency data to the emergency data recipient.

### Emergency Flow Management System

In some embodiments, as depicted in FIG. 1, the emergency response data platform (ERDP) includes an emergency flow management system (EFMS) 114. Generally, the EFMS functions to provide digital connectivity to emergency services to devices and applications otherwise unable to access them. Using the EFMS, an administrator of a device or application can access an emergency flow editor (also referred to as an “emergency console”), select a default emergency flow or define their own custom emergency flow, and receive an emergency flow trigger script including an emergency flow identifier (also referred to as an “emergency flow ID”) unique to their chosen emergency flow. The emergency flow trigger script can then be quickly and easily integrated into the administrator's device or application. When the emergency flow trigger script is executed by the administrator's device or application, an electronic notification including the emergency flow ID is transmitted to an endpoint provided by the EFMS, which prompts the EFMS to execute the associated emergency flow chosen by the administrator.

An emergency flow may prompt the EFMS to perform a variety of functions, including (but not limited to) transmitting a notification to an emergency contact, transmitting a request for emergency service to an emergency service provider (ESP), establishing an emergency communication bridge to facilitate a Voice over Internet Protocol (VoIP) call between two or more participants, transmitting emergency data to one or more emergency data recipients, or any combination thereof, depending on the administrator's intended use case. In some embodiments, the emergency console provides a set of default emergency flows to choose from. In some embodiments, the emergency console provides a graphical user interface (GUI) that a user (e.g., an administrator of a device or application) can user to create a custom emergency flow. In some embodiments, the GUI of the emergency console allows a user to create a custom emergency flow by dragging and dropping (or otherwise manipulating) graphical representations of emergency flow building blocks into various arrangements, which prompts the EFMS to automatically generate an emergency flow according to the arrangement of the emergency flow building blocks. In some embodiments, an emergency flow building block is defined by a short script (e.g., a compilation or block of written programming commands), written in a programming language, that contains instructions for executing one or more functions. In some embodiments, when a user arranges one or more emergency flow building blocks within the GUI of the emergency console, the EFMS generates an emergency flow according to the arrangement of the one or more emergency flow building blocks by compiling the short scripts defining each of the one or more emergency flow building blocks into a single emergency flow script. In some embodiments, the emergency console allows a user to edit or create an emergency flow script directly (e.g., without the use of graphical representations of emergency flow building blocks, such as by inputting written program commands directly into a programming language input field).

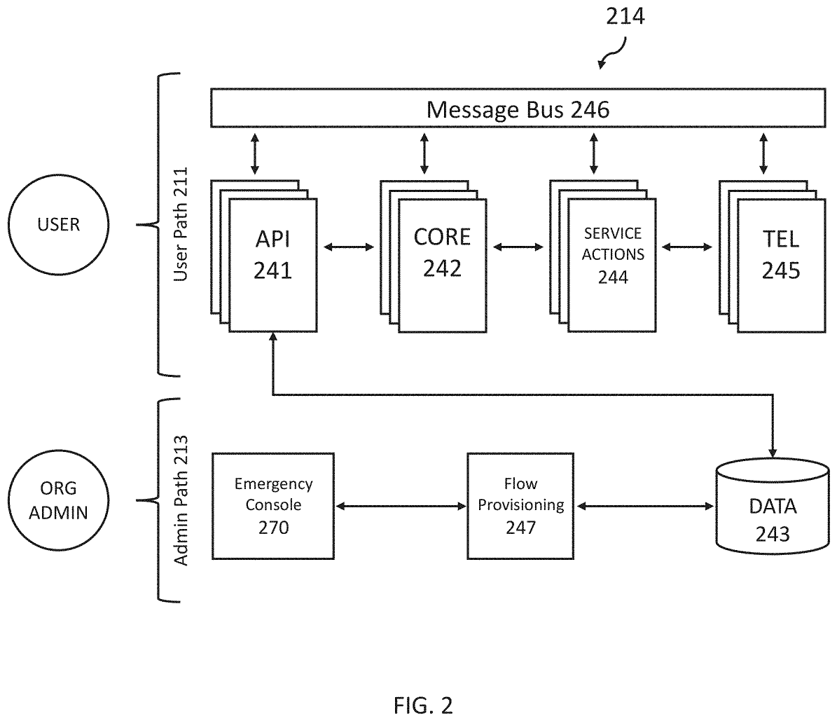

FIG. 2 depicts an embodiment of an emergency flow management system (EFMS). As depicted in FIG. 2, in some embodiments, the EFMS 214 contains two pathways: an administrator pathway 213 (admin path) and a user pathway 211 (user path). The admin path 213 is initiated by an administrator of a device or application, as described above. In the admin path, the administrator accesses an emergency flow editor 270 to configure an emergency flow to fir the needs of the administrator's product or service. In some embodiments, in the admin path, an emergency flow provisioning module 247 compiles the emergency flow into an emergency flow script, assigns an emergency flow ID to the emergency flow script, and stores the emergency flow script within a data module 243. Finally, the EFMS provides the administrator with an emergency flow trigger script including the emergency flow ID, which the administrator can integrate into their device or application. The user path 211 is initiated by a user, or a device associated with a user, of the product or service provided by the administrator. In some embodiments, in the user path, the API module 241 receives an electronic notification including the emergency flow ID from a device or application that the administrator has integrated the emergency flow trigger script into. In some embodiments, the API module 241 then references the data module 243 with the emergency flow ID to identify the emergency flow script corresponding to the emergency flow ID and delivers the emergency flow script to the core module 242 for execution. In some embodiments, the core module 242 then employs the service actions module 244 and the telephony module 245 to execute the various functions included in the emergency flow script. In some embodiments, the API module 241, the core module 242, the service actions module 244, and the telephony module 245 are separately and simultaneously in communication with the message bus 246, which facilitates and coordinates synchronous and asynchronous communication functions (e.g., a communication bridge, text messages, etc.) between the modules and various users and accounts (e.g., a user, emergency contacts, emergency responders, etc.). In some embodiments, the electronic notification including the emergency flow ID also contains emergency data, such as user data, location data, or any other additional data, according to the administrator's use case. In some embodiments, emergency data ingested by the EFMS 214 is received and shared by the emergency clearinghouse, as described above.

After an emergency is triggered, the EFMS 214 may be utilized to ingest the emergency data and follow a pathway based on the emergency data, user permissions, ESP authorization and bandwidth. In some embodiments, the EFMS is used to call the user to confirm the emergency. In some embodiments, the user may submit audio or video files regarding the emergency for sharing with emergency personnel. In some embodiments, the EFMS may notify emergency contacts via various methods such as SMS text messages, SMS data messages, phone call, push notifications, email, or other type of internet-based communications.

### Emergency Response Application

As mentioned above, in some embodiments, data and information is shared between the emergency response data platform (ERDP) and an emergency service provider (ESP) through an emergency response application. In some embodiments, as described in further detail below, the emergency response application may additionally be provided to an ESP to: a) facilitate communications between the ESP and an emergency caller (e.g., a person requesting emergency assistance) or b) facilitate communications between the ESP and one or more other ESPs. In some embodiments, the emergency response application is a software application either installed on a computing device at the ESP or accessed via the internet through a web browser on the computing device (e.g., the emergency response application is hosted on a cloud computing system by the ERDP). In some embodiments, the emergency response application functions to both facilitate a two-way communication link between the ERDP and the ESP and visualize data (e.g., emergency data) received by the ESP from the ERDP. The emergency response application optionally includes various components, such as a frontend application (hereinafter “graphical user interface” or “GUI”), a backend application, an authorization module, and a user database. In some embodiments, the emergency response application additionally or alternatively includes a credential management system or a geofence system (which may include or be otherwise communicatively coupled to a credentials database or a geofence database). In some embodiments, the credential management system and the geofence system are external to the emergency response application and communicatively coupled to the emergency response application (e.g., the credential management system or geofence system can be housed or hosted on a cloud computing system by the ERDP). Any or all of the components of the emergency response application may be hosted on a cloud computing system by the ERDP, a computing device at an ESP, or some combination thereof.

In some embodiments, the emergency response application is a webpage or web application that can be accessed through an internet or web browser. In such embodiments, the emergency response application can be quickly and easily integrated into the systems used by emergency service providers (ESPs), such as public safety answering points (PSAPs), because accessing and using emergency response application requires no additional software or hardware outside of standard computing devices and networks. As previously discussed, one of the greatest hinderances that PSAPs face in providing emergency assistance to people experiencing emergency situations is in acquiring accurate locations of the emergencies and the people involved, because PSAPs are currently typically limited to verbally asking for and verbally receiving locations from callers. In some embodiments, the clearinghouse is capable of receiving accurate locations (as well as additional emergency data, as described above) from electronic devices such as smartphones and delivering the accurate locations to the appropriate PSAPs during emergency situations. Therefore, it is advantageous to provide the emergency response application to PSAPs in the form of a webpage accessible through a standard web browser, in order to provide the potentially life-saving information stored within the clearinghouse to those capable of providing emergency assistance as quickly and easily as possible. However, in some embodiments, the emergency response application is a software application installed on a computing device at an ESP. The emergency response application may be provided by the ERDP or by a third-party.

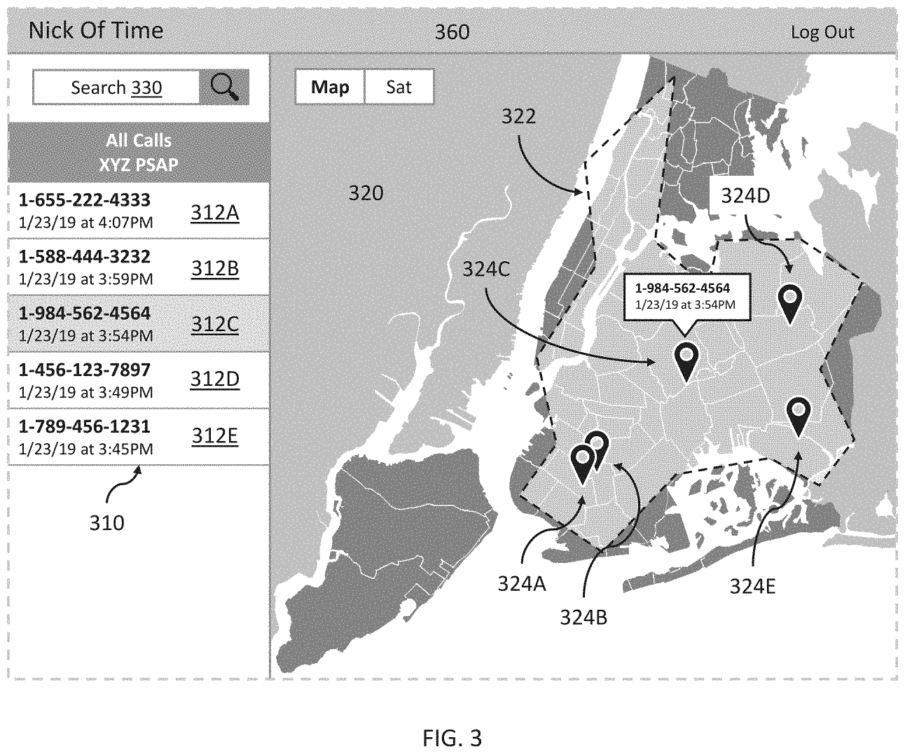

FIG. 3 illustrates an embodiment of a graphical user interface (GUI) provided by an emergency response application 360. In some embodiments, the GUI provides interactive elements that allow a user at an ESP to receive data from the ERDP, visualize data received from the ERDP, and transmit data to the ERDP. For example, in some embodiments, the GUI includes an entry field 330 through which a user can submit a device identifier, such as by typing or pasting the device identifier into the entry field 330. In some embodiments, after submitting a device identifier through the entry field 330, the user can prompt the emergency response application to generate and send an emergency data request by selecting a search button. The emergency response application 360 then generates an emergency data request including the device identifier and any other necessary information (e.g., a temporary access token) and transmits the emergency data request to the ERDP. The ERDP can then return any available emergency data associated with the device identifier to the emergency response application 360, as described above and below. In another example, in some embodiments, the emergency response application 360 can automatically receive emergency data from the ERDP for emergencies relevant to an ESP (e.g., emergencies located within the jurisdiction of the ESP) without requiring a user to generate an emergency data request, as described above and below. After receiving emergency data from the ERDP, the emergency response application 360 can then visualize the emergency data within the GUI of the emergency response application 360. For example, in some embodiments, the emergency response application 360 includes a list of incidents 310 and an interactive map 320, as illustrated by FIG. 3. As shown, in some embodiments, when the emergency response application 360 receives a location (e.g., an emergency location) and a device identifier associated with an emergency occurring within the jurisdiction 322 of the receiving ESP, the emergency response application 360 displays the location associated with the emergency within the interactive map 320 as a location marker 324 (also referred to as an “incident location”) and displays the device identifier associated with the emergency within the list of incidents 310 as an incident 312.

In addition to emergency locations, the emergency response application 360 can receive and visualize numerous types of emergency data from the ERDP. For example, the emergency response application 360 can receive additional data regarding an emergency, such as demographic or medical data associated with a person involved in the emergency (e.g., an emergency caller). In another example, the emergency response application 360 can receive data from sensors associated with the emergency, such as heartrate data collected by a sensor on an emergency caller's smartwatch. Or, for example, the emergency response application 360 can receive data regarding emergency response assets available for an emergency, as described below. In some embodiments, the emergency response application receives and visualizes messages received from emergency callers or other ESPs, as described below. The emergency response application 360 can visualize any emergency data received from the ERDP within the GUI of the emergency response application.

### Emergency Data Transmission

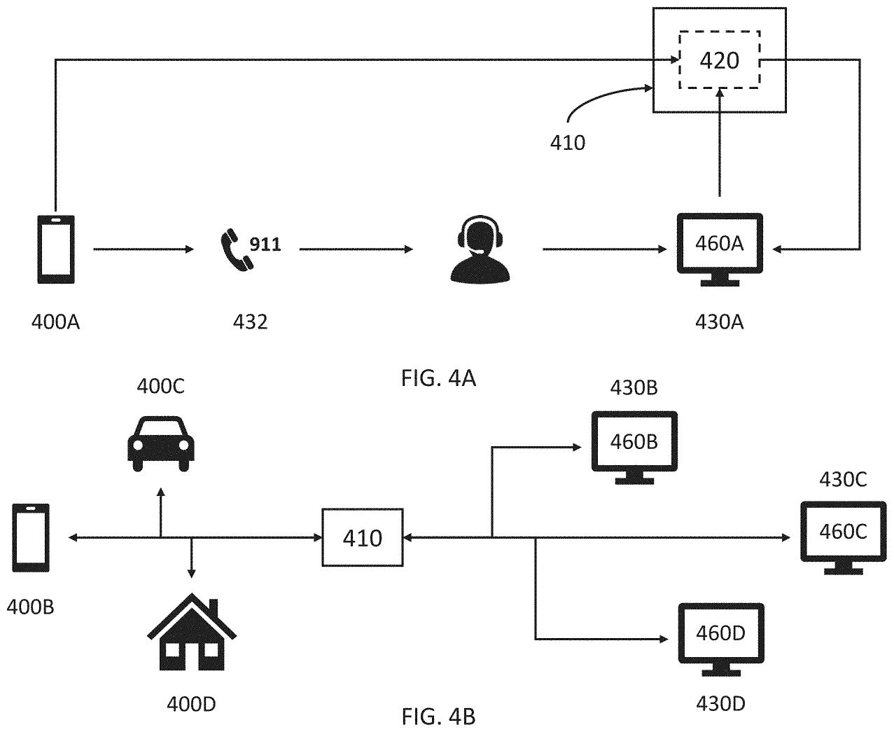

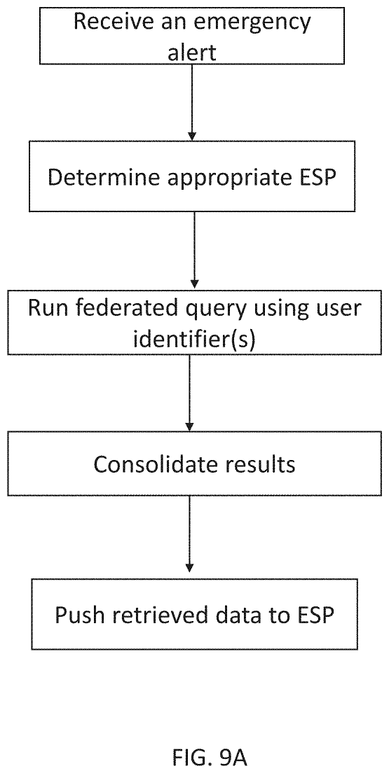

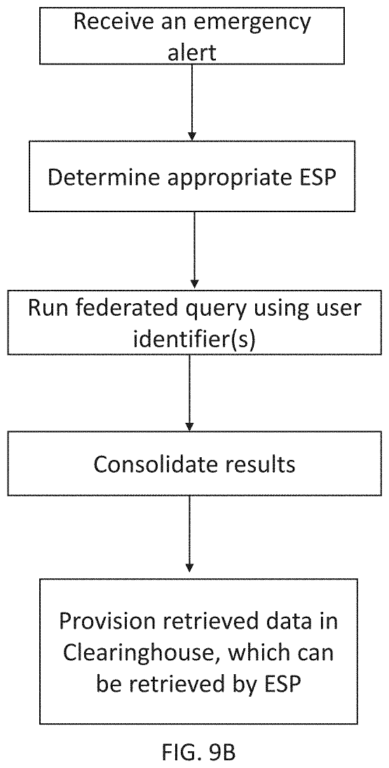

FIGS. 4A and 4B depict systems and processes for receiving and transmitting emergency data by an emergency response data platform in accordance with some embodiments of the present disclosure. As described above, in some embodiments, an emergency response data platform (ERDP) maintains a clearinghouse that obtains and shares emergency data to aid emergency service providers (ESPs) in responding to emergencies. For example, as depicted in FIG. 4A, during an emergency, an ESP 430A can send a query for emergency data (also referred to as an “emergency data request”) to the ERDP 410 (e.g., through an emergency response application 460A, as described above) for a particular emergency, and, in response, the ERDP 410 can send any available emergency data associated with the emergency back to the ESP 430A (such as through emergency response application 460A). In some embodiments, as described above, the emergency response application 460A includes an identifier associated with an emergency alert in the emergency data request. The ERDP 410 can then use the identifier associated with the emergency alert to retrieve emergency data associated with the emergency alert from the clearinghouse 420. For example, as described above, an ESP 740A (e.g., a public safety answering point (PSAP)) can receive an emergency alert in the form of a 9-1-1 phone call 432 (representative of an emergency or potential emergency) from a mobile phone 400A associated with a phone number (e.g., (555) 555-5555). The ESP 430A can then send an emergency data request including the phone number (i.e., the identifier associated with the emergency alert) to the ERDP 410, which can then retrieve any emergency data within the clearinghouse 420 associated with the phone number and return the available emergency data to the requesting ESP 430A. This process of returning emergency data to an ESP in response to an emergency data request is referred to as “pulling” emergency data from the clearinghouse. An illustrative method is depicted in FIG. 9B.

As described above, in some embodiments, the emergency response data platform (ERDP) can “push” emergency data from the Emergency Clearinghouse to emergency service providers (ESPs), such as by using an emergency data subscription system (hereinafter, “subscription system”). FIG. 4B depicts a flow diagram of a process for pushing emergency data from the Emergency Clearinghouse to one or more ESPs. In some embodiments, a member of an ESP (e.g., a PSAP staff member) logs into the emergency response application 460B at an ESP system 430B (e.g., a computing device associated with the ESP) by accessing the emergency response application 460B (e.g., by navigating to the emergency response application 460B through a web browser) and submitting their login information through the GUI of the emergency response application 460B. In some embodiments, when the ESP member logs into the emergency response application 460B by submitting their login information, the emergency response application 460B or ERDP 410 then determines an ESP account ID associated with the ESP member's account and establishes a persistent or active communication link (e.g., a websocket connection) with the ESP system 430B, thereby automatically subscribing the ESP console to the ESP account ID for the duration of their login session. Then, as described above, when the ERDP 410 receives an emergency alert including a location (e.g., when an emergency call is made from an electronic device 400B and sends an emergency alert to the ERDP 410 including a location generated by the electronic device 400B), the ERDP 410 retrieves a geofence associated with every ESP registered with the ERDP 410 and determines if the location falls within any of the geofences. In response to determining that the location falls within a geofence associated with the ESP associated with the ESP account ID, the ERDP 410 then associates the location with the ESP account ID, determines if there are any active or persistent communication links between the ERDP 410 and any computing devices subscribed to the ESP account ID. In this instance, because the ESP system 430B is subscribed to the ESP account ID and actively linked to the ERDP 410 through the persistent or active communication link, the ERDP 410 automatically pushes (e.g., from the clearinghouse) the emergency alert or emergency data associated with the emergency alert (e.g., the location, a phone number, etc.) to the ESP system 430B for display within the emergency response application 460B. In some embodiments, emergency alerts or emergency data associated with emergency alerts that have been pushed to an ESP are displayed within a jurisdictional awareness view, as described below.

For example, ESP system 430B and ESP system 430C are two different ESP consoles associated with the same ESP (e.g., two computing devices at the same public safety answering point), PSAP A. ESP system 430D is associated with a second ESP, PSAP B. Using this system, PSAP call-takers can access and log into the emergency response application 460 (emergency response application 460D-460D) at each of the three ESP system (ESP systems 430B-430D), thereby establishing three separate active communication links, one active communication link between the ERDP 410 and each of the three ESP consoles. The ESP consoles are automatically subscribed by the ERDP 410 to the ESP account IDs associated with their respective ESPs (ESP ID A for PSAP A and ESP ID B for PSAP B). Both PSAP A and PSAP B are associated with only one geofence, geofence A and geofence B, respectively. Geofences A and B do not overlap, especially for primary agencies which have authoritative jurisdiction. The geofences have previously been tagged within the ERDP 410 with their respective ESP account IDs (e.g., during a registration process for the emergency response application). It is contemplated that an ESP (e.g., a PSAP) may be associated with one or more geofences or sub-geofences. For example, the area where emergency calls may be made versus another area where text messages to 911 may be made. In another example, an ESP agency may have a specific geofence for responding to specific type of emergency, e.g., medical emergency geofence.

When an emergency call is later made from communication device 400B, the communication device 400B generates a first emergency alert including a first location of the communication device 400B and transmit the first emergency alert to the ERDP 410. When the ERDP 410 receives the first emergency alert, the ERDP 410 retrieves some or all of the geofences stored within the ERDP 410 and determines if the first location falls within any of the geofences stored within the ERDP 410. In the event that the ERDP 410 determines that the first location falls within geofence A, associated with PSAP A, the ERDP 410 tags the first location with the ESP account ID associated with geofence A, ESP ID A. The ERDP 410 then determines if there are any active communication links between the ERDP and any ESP consoles subscribed to ESP ID A and automatically pushes (e.g., from the clearinghouse) the first emergency alert to those ESP consoles. In this example, both ESP system 430B and ESP system 430C are subscribed to ESP ID A, so the ERDP 410 automatically pushes the first emergency alert to both ESP system 430B and ESP system 430C for display within emergency response applications 460B and 460C, respectively, such as through a jurisdictional awareness view (as described below). The first location does not fall within geofence B, because geofence A and geofence B do not overlap, and as a result, the first emergency alert is not pushed to ESP system 430D, even though an active communication link has been established between the ERDP 410 and ESP system 430D.

The ERDP 410 may then receive an emergency alert from electronic device 400D (e.g., a home security system) including a second location of the electronic device 400D. When the ERDP 410 receives the second emergency alert, the ERDP again retrieves some or all of the geofences stored within the ERDP 410 and determines if the second location falls within any of the geofences stored within the ERDP 410. In this example, the ERDP 410 determines that the second location falls within geofence B, associated with PSAP B. In response, the ERDP 410 tags the second location within the ESP account associated with geofence B, ESP ID B and automatically pushes the second emergency alert to ESP system 430D for display within emergency response application 460D, because ESP system 430D has an active communication link established with the ERDP 410 and ESP system 430D is subscribed to ESP ID B. The ERDP 410 does not push the second emergency alert to ESP system 430B or ESP system 430C. Although ESP system 430B and ESP system 430C have active communication links established with the ERDP 410, they are not subscribed to ESP ID B, and geofence A and geofence B do not overlap, meaning the second location does not fall within geofence A. Shortly thereafter, the ERDP 410 may receive an emergency alert from electronic device 400C (e.g., an intelligent vehicle system) including a third location of the electronic device 400C. The ERDP 410 determines that the third locations falls within geofence A (like the first location included in the first emergency alert) and thus automatically pushes the third emergency alert to both ESP system 430B and ESP system 430C for display within emergency response application 460B and 460C. In some embodiments, emergency response application 460B and emergency response application 460C display the first emergency alert and the third emergency alert simultaneously, such as through a jurisdictional awareness view, as described below.

### Jurisdictional Awareness View

In some embodiments, the systems, applications, servers, devices, methods, and media of the instant application provide a jurisdictional awareness view within the emergency response application. In some embodiments, the jurisdictional awareness view enables an ESP to view one or more ongoing or recently received emergency alerts (e.g., emergency calls) within one or more geofenced jurisdictions. Although not shown, ectronic emergency alerts based on alarms or sensors, activation of a panic button, etc. may be displayed in a similar fashion wherein the emergency alerts are not emergency calls. FIG. 3 illustrates the jurisdictional awareness view displayed within the emergency response application, in accordance with one embodiment of the present disclosure. In some embodiments, the jurisdictional awareness view includes a list of incidents 310 that displays one or more incidents 312 associated with one or more device identifiers (e.g. phone numbers, IP addresses). In some embodiments, the jurisdictional awareness view additionally or alternatively includes an interactive map 320 that displays one or more incident locations 324 associated with the one or more incidents 312 associated with the one or more device identifiers, as described below. In some embodiments, the jurisdictional awareness view displays incidents and incident locations only for emergencies occurring within the jurisdiction 322 of the ESP at which the emergency response application 360 is being accessed.

For example, as illustrated in FIG. 3, an ESP may access an emergency response application 360 provided by the ERDP. In this example, the ERDP has pushed emergency data associated with five different emergency alerts to the ESP (as described above) through the emergency response application 360. Accordingly, the emergency response application 360 displays five different incidents 312 (e.g., incidents 312A-312E) within the list of incidents 310 and five corresponding incident locations 324 (e.g., incident locations 324A-324E) within the interactive map 362. As illustrated by FIG. 3, in some embodiments, incidents 312 and incident locations 324 may be selected or hovered over to highlight a particular incident 312. In this example, incident 312C and its corresponding incident location 324C have been selected and highlighted. In some embodiments, selecting a particular incident 312 or corresponding incident location 324 prompts the emergency response application 360 to display additional information associated with the particular incident 312 (e.g., additional emergency data or information associated with the emergency alert for which the particular incident 312 was created). Because the jurisdictional awareness view can show an ESP numerous incidents 312 occurring within the jurisdiction 322 of the ESP simultaneously, the jurisdictional awareness view can provide the ESP with situational awareness that the ESP otherwise would not have. For example, with the knowledge that incidents 312A and 312B originated in close proximity and at approximately the same time, an ESP personnel (e.g., a call taker at a public safety answering point) can determine that the two incidents may be related. An illustrative method is depicted in FIG. 9A.

### Emergency Profiles (EP)

An emergency profile (EP) is a data record that contains information about the user such as, biographical, demographical, medical, permissions, etc., that may be used to provide efficient emergency response. In some embodiments, the information in the EP may be entered by a user before the emergency. In some embodiments, the user may be prompted to enter the EP information when the emergency is on-going. For example, once an emergency has been triggered, a user may be prompted to provide information about his or her Covid symptoms or vaccination records before emergency responders arrive on the scene. In some cases, the user may enter information about their Covid status such as symptoms, test results, or vaccination status when providing information for the emergency profile. In some instances, the user is able to upload corroborating evidence such as a photo of a vaccination card or an identifier allowing such information to be queried against a third party system such as a public health database. In some embodiments, the EP contains information from two or more sources via a federated query, as described below.

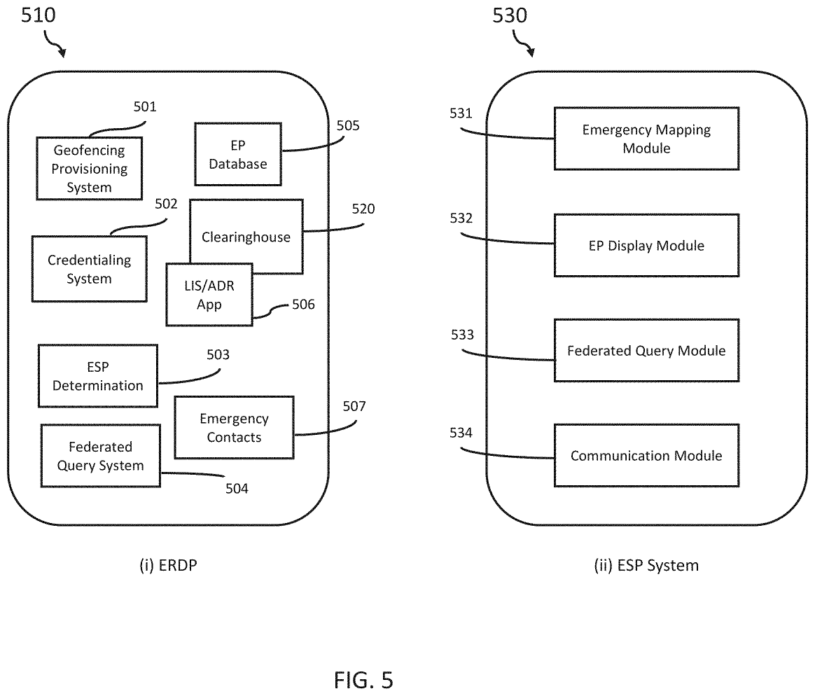

For illustration, FIG. 5 depicts components of the (i) ERDP and (ii) ESP system. In some embodiments, the ERDP 510 includes one or more EP databases 505, a clearinghouse 520 associated with location and additional data querying Apps 506, and emergency contacts procedure 507, a geofence provisioning system 501, an ESP credentialing/authorization system 502, ESP determination module 503, and a federated query system 504. In some embodiments, the clearinghouse 520, as described in further detail below, is an input/output (I/O) interface configured to manage communications and data transfers to and from the ERDP 510 and external systems and devices. In some embodiments, the clearinghouse 520 includes a variety of software and hardware interfaces, for example, a web interface, a graphical user interface (GUI), and the like. The clearinghouse 520 optionally enables the ERDP 510 to communicate with other computing devices, such as web servers and external data servers (not shown). In some embodiments, the clearinghouse 520 facilitates multiple communications within a wide variety of networks and protocol types, including wired networks, for example, LAN, cable, etc., and wireless networks, such as WLAN, cellular, or satellite. In some embodiments, the clearinghouse 520 includes one or more ports for connecting a number of devices to one another or to another server. In some embodiments, the clearinghouse 520 includes one or more sub-clearinghouses, such as location clearinghouse and additional data clearinghouses (not shown).

The ERDP 510 additionally includes an emergency profile (EP) database 505 that collects and stores user information (e.g., personal information, demographic information, medical information, location information, etc.). In some embodiments, users can submit user information through a website, web application, or mobile application, such as during a registration process for an emergency profile (EP). In some embodiments, user information stored within the EP DB 505 is received by the ERDP 510 from one or more third-party servers or databases, as described below, based on one or more queries. In some embodiments, user information stored within the emergency profile DB 505 is associated with an identifier of the user (e.g., name, phone number, email address, social security, address, etc.). In some embodiments, an EP data analysis module (not shown) processes retrieved data from one or more third-party servers into a standardized format for the ESP. In some embodiments, the encryption/decryption tools (not shown) can be used to encrypt and decrypt private data (e.g., medical data) for privacy and security measures.

Data obtained from one or more third-party servers or sources can be processed to extract relevant data parameters. For example, third party data may include medical information such as electronic medical records, which can include a wide range of formats such as doctor's notes or lab testing results. Accordingly, in some embodiments, provided herein are systems and methods that utilize algorithms to extract data parameters or features from the data. The algorithms can include natural language processing algorithms. In certain instances, a software module is configured to apply a machine learning algorithm for carrying out natural language processing on the retrieved data (e.g., lab tests/results, doctor visit summaries).

The ESP system may be a public or private agency that responds to emergencies, such as PSAP. The ESP system may be a call handling or dispatching system with devices such as workstations, mobile devices, etc. In some embodiments, an ESP system 530 (e.g., public safety answering point (PSAP)) that includes one or more of a display, a user interface, at least one central processing unit or processor, a network component, an audio system (e.g., microphone, speaker and/or a call-taking headset), and a computer program such as an EP Display Program (not shown). In some embodiments, the EP Display Program comprises one or more software modules.

In some embodiments, the ESP system 530 comprises an emergency mapping module 531, an EP display module 532 (for displaying emergency data in the EP), federated query display module 533 (for displaying emergency data with source information, reliability score, age, etc. or for querying additional sources that have not been automatically queried), communication module 534. In some cases, data retrieved from a federated query may be filtered or sorted for relevant & reliable information. For example, the data may be obtained from a variety of sources and can include too much information so as to be overwhelming or hinder the emergency response. Therefore, in some cases, the data is sorted or filtered for location information and relevant medical information. Relevance can be based on the data field. For example, the type of emergency may be known (e.g., indicated in the emergency alert). In the case of a medical emergency, in some cases, medical information such as preexisting conditions, Covid status, and biomonitoring data (e.g., heart rate from a smartwatch) may be determined to be relevant. One or more rules defining relevance may be applied to identify relevant data. The rules can be pre-defined, for example, indicating certain types or sources of data as relevant for certain categories of emergencies. In some cases, the rules limit the amount of data that can be displayed to avoid cluttering the display. The rules can also be used to prioritize data or categories of data such that when the amount of relevant data available exceeds a limit on what can be displayed, the higher priority data or categories of data are made available for display while lower priority data or categories of data are not shown.

In some embodiments, the ESP system 530 includes a communication module that allows the emergency personnel to contact the user, one or more emergency contacts, medical providers, behavioral health providers, police, fire, etc. The ESP system 530 may send communication by known methods, e.g., a phone call, SMS, email, or internet-based messaging.

In some embodiments, location and additional data is displayed for emergency service providers (e.g., police, fire, medical, etc.) and/or responders on their devices. It is contemplated that responder devices have optionally installed a responder device program (not shown) similar to mapping module 531, EP display module 532, federated query module 533 and communication module 534.

In some embodiments, an EP portal is provided for users to enter user data before an emergency has occurred. The advantage of entering the information in case of emergencies is that the user can enter the information without rushing and also note critical data such as medical conditions, behavioral conditions that require special procedures. In addition, the user can provide consent to share specific data, which may be sensitive or protected only in the case of an emergency. It is contemplated that user permissions can be given based on detailed user preferences. Some data may have to be shared and the user may not be given a chance to opt out, such as user name, address, contact information.

In some embodiments, the EP database(s) comprise one or more database systems including, by way of non-limiting examples, relational, non-relational, object oriented, associative, and XML database systems. In some embodiments, the database(s) 235 is a relational database with time-stamped columns such as user name, phone number (or call back number), location data, sensor data, saved user data, and other information. In some embodiments, the data is saved in a non-relational or NoSQL database associated with a time-stamp and a user ID (such as phone number, user's name, or device ID, etc.).

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

In some embodiments, as described above, emergency data includes locations and additional data. In some embodiments, emergency data includes one or more emergency data categories (also referred to as “data categories”). In some embodiments, the emergency data categories include: service data reference, full name, email, emergency contacts, addresses, language, occupation, phone numbers, websites, gender, height, weight, ethnicity, profile picture, allergies, medical conditions, medications, disabilities, blood type, medical notes, birthday, and additional comments. In some embodiments, emergency data categories are tagged with tags for specific types of data such as “demographics” or “medical data.” For example, in some embodiments, gender, height, weight, ethnicity, profile picture (image-url) are tagged as demographic data. In some embodiments, medical data protected under HIPAA and other laws are tagged as “HIPAA” or “private.” In some embodiments, medical data includes information on one or more of allergies, medical condition(s) or illness(es), medication(s), disabilities, blood type, medical note(s), and other medical information. In some embodiments, medical information protected under HIPAA are encrypted and/or anonymized. In some embodiments, some data are tagged as “general” or another similar tag, wherein access is not specifically restricted.

In some embodiments, the categories of data include one or more of altitude, caller ID, cell carrier, device IMEI, device model, device number, emergency number, location accuracy, location altitude, location latitude, location longitude, location time, place address, source, time, registered address, registered engine, and uncertainty radius.

In some non-emergency situations, there is a need to access location data, user data, emergency data or sensor data. For example, in some embodiments, a user of an electronic device grants authorization to family members to access location data for the user. Accordingly, when a family member requests location data for a user, access is granted if there is proper authorization. As another example, in some embodiments, a taxi operations company requests and obtains location data of one or more fleet members to keep track of its vehicles (e.g., via onboard vehicle console or terminal).

In other embodiments, the user is prompted to provide, confirm or give consent to access emergency data after the emergency has commenced. During an emergency, specific information may be deemed “critical” and the user is prompted to provide or confirm the information. For example, a user may be prompted to confirm the location of the emergency and how many people are involved. Also, during a medical emergency, a user may be prompted to confirm his or her blood-type, medical conditions, allergies, etc.

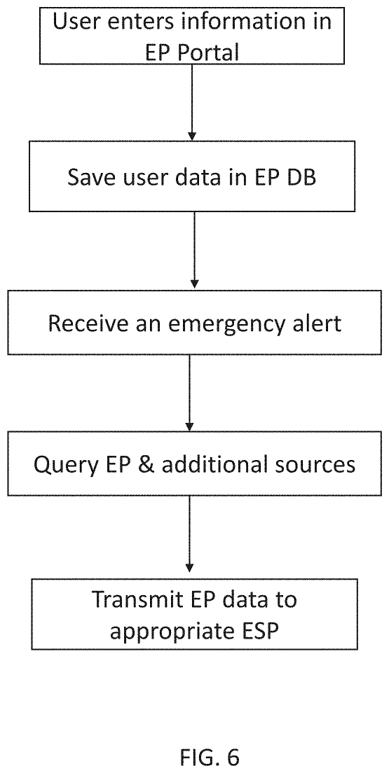

In some embodiments, the EP portal is a web-hosted platform, a downloadable app, built-in application or part of the operating system. FIG. 6 depicts an illustrative method for generating and sharing an emergency profile (EP).

Using the EP portal, a user is prompted to enter information about themselves (or a caregiver about a patient). User-entered data may be saved in an EP DB (e.g., EP DB 505 shown in FIG. 5). When an emergency alert is received by the ERDP, it may be in the form of a notification about the initiation of an emergency communication session (e.g., 911 call). The emergency alert contains a user identifier (e.g., a phone number, user name, location, etc.) for querying the EP DB. Additional sources can also be queried to get more information. Finally, relevant and reliable EP data is transmitted to an appropriate ESP.

As used herein, an “emergency service provider” (ESP) is a public or private organization or institution responsible for providing emergency services. For example, in some embodiments, an EDC (e.g., a public safety answering point (PSAP)), a fire department, a police department, and a hospital may all be considered emergency service providers. In some embodiments, an emergency responder is a member of an ESP. In some embodiments, an ESP personnel is a person who works at an ESP. For example, an ESP personnel may be a call-taker at a PSAP or a first responder at a fire department.

As used herein, a “emergency responder” refers to any person or persons responsible for addressing an emergency situation. In some embodiments, a first responder refers to government personnel responsible for addressing an emergency situation. In some embodiments, a first responder is responsible for a particular jurisdiction (e.g., a municipality, a township, a county, etc.). In some embodiments, a first responder is assigned to an emergency by an emergency dispatch center. In some embodiments, a first responder responds to a request for emergency assistance placed by a user via a user communication device. In some embodiments, a first responder includes one or more fire fighters, police officers, emergency medical personnel, community volunteers, private security, security personnel at a university, or other persons employed to protect and serve the public and/or certain subsets of the population.

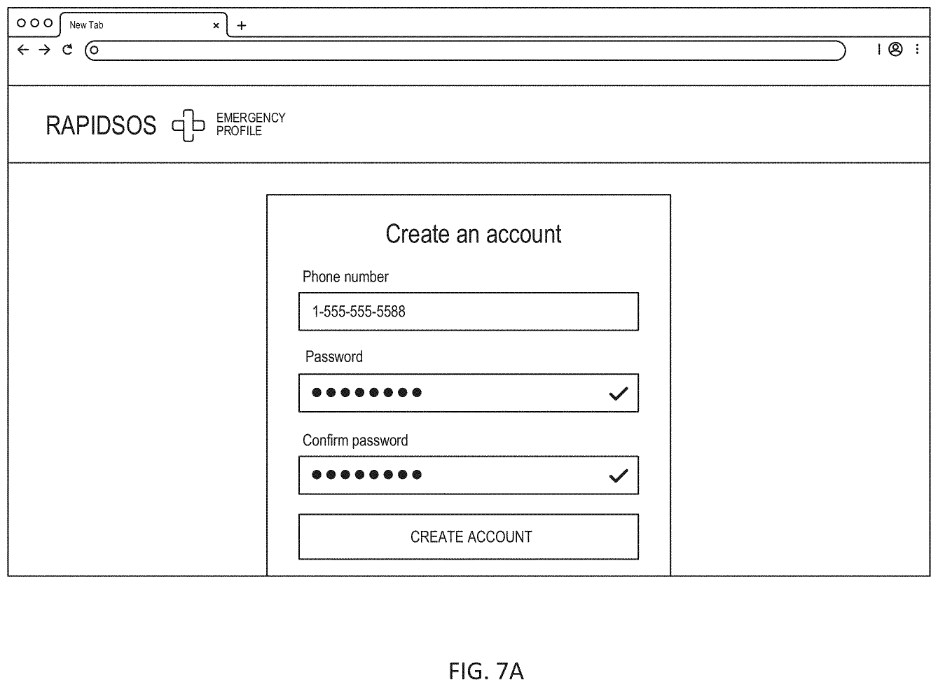

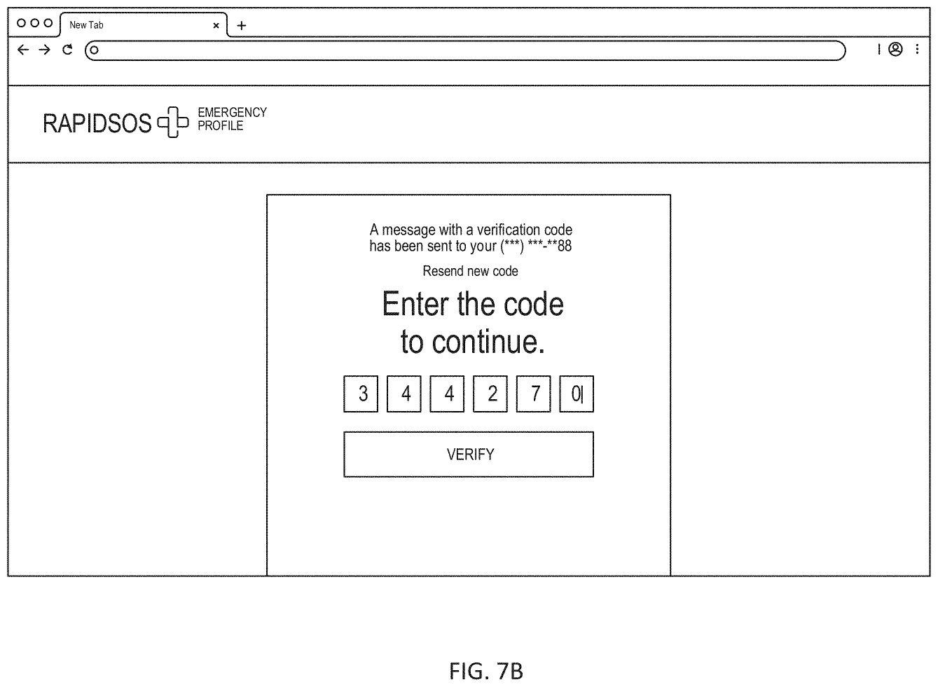

FIGS. 7A & 7B depict interfaces for providing a portal for users to provide information for an EP. As shown, a user can use a web-portal for registering for an emergency profile. For example, log-in and password and a 2-step verification may be required using a user phone for registering. In a similar way, emergency contacts may be required to verify their devices for receiving notification and status updates.

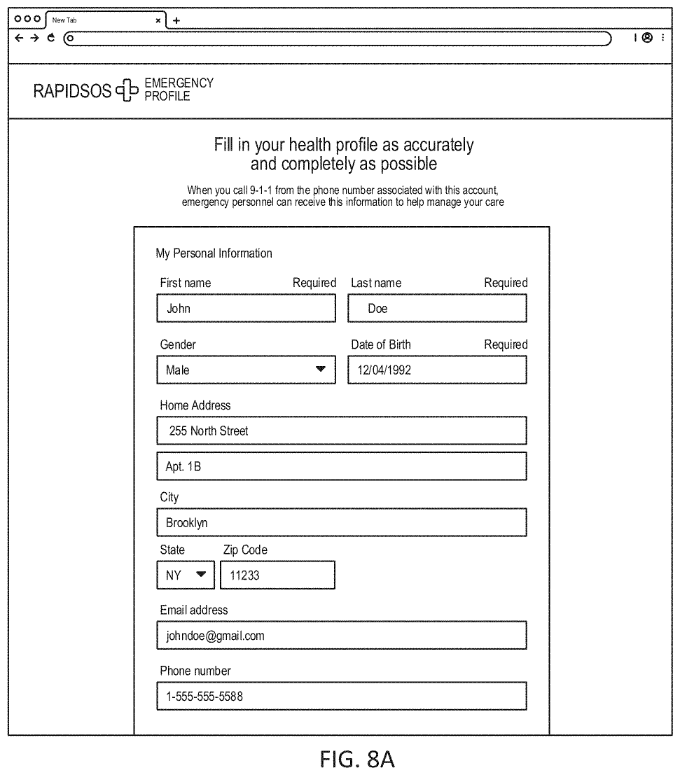

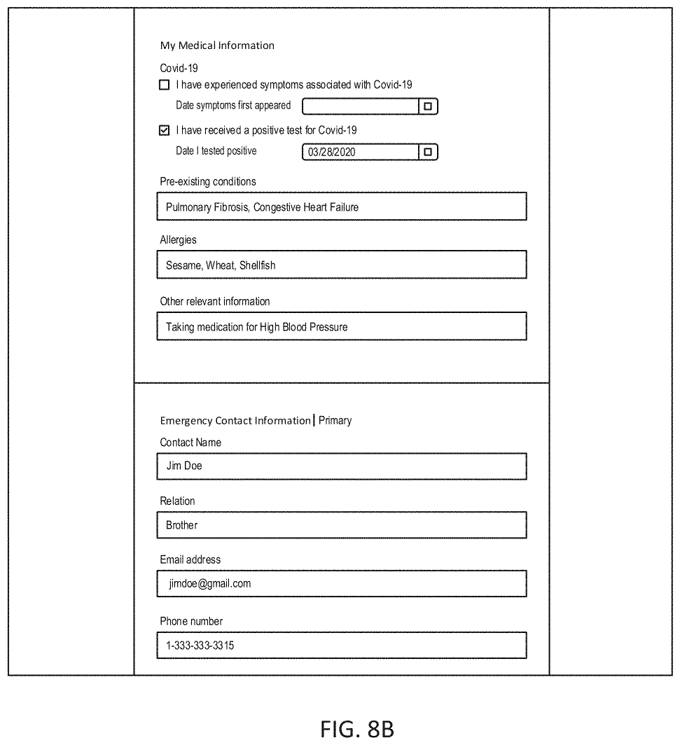

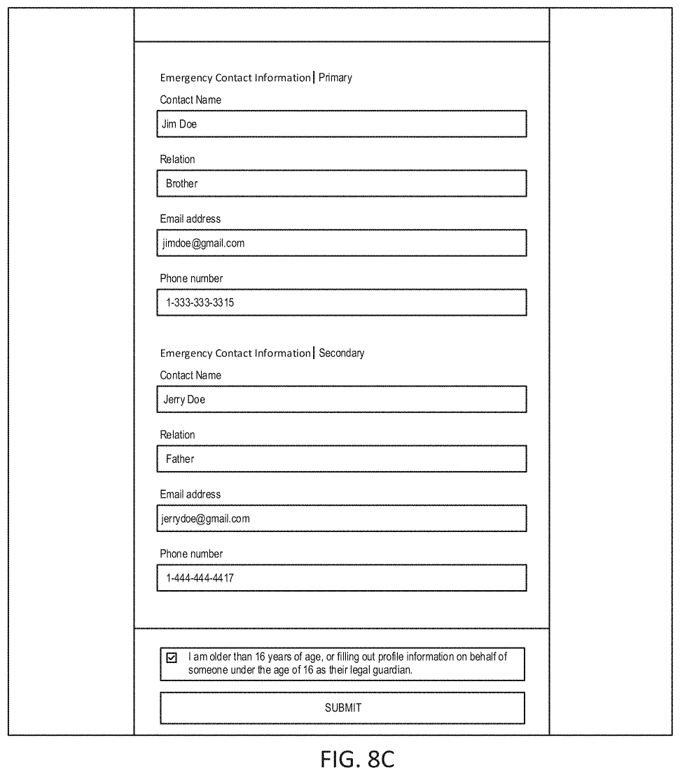

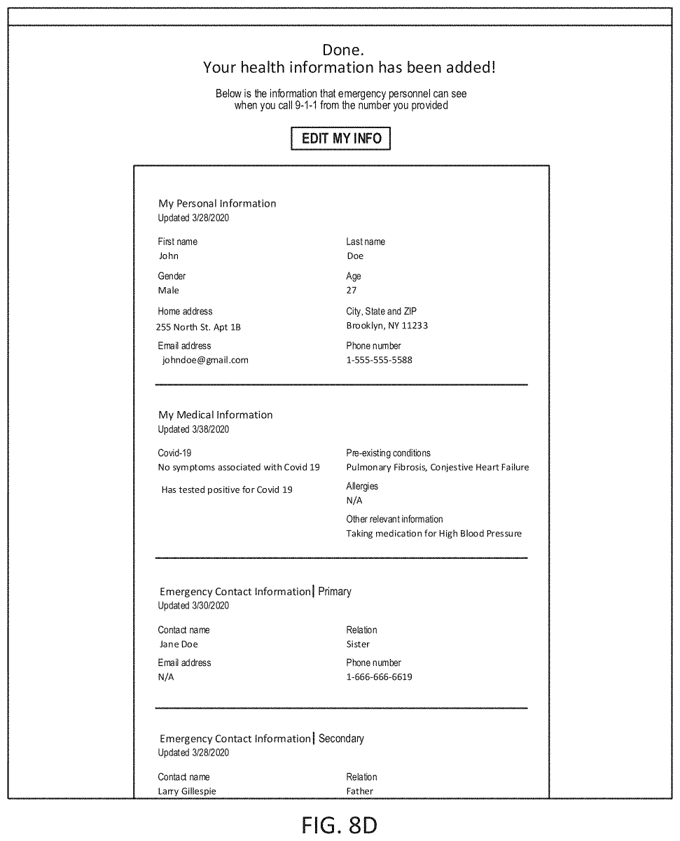

FIGS. 8A-D illustrates interfaces for a user to provide information for an EP. For example, a user may be prompted to provide information for the emergency response such as name, address, email, phone number, date of birth, gender, etc. In some embodiments, some of the entries may be gathered based on sensor data from associated devices (e.g., resting heart rate reading from a wearable device). In some embodiments, emergency data may be gathered from one or more third-party sources. For example, data from a medical database may be used for obtaining blood type, drug prescriptions, etc.

The interfaces shown are meant to be non-limiting illustrative embodiments and it is contemplated that other information may also be obtained from the users such as social security number, work address, alternate number, alternate email, medical insurance information, primary physicians, blood type, etc.

As shown in FIG. 4B, a user may be prompted to provide symptoms for a specific disease, e.g., Covid-19, AIDS, SARS. Test results may also be entered or obtained from public health databases into the emergency profile. In addition, databases may be queried for vaccine information including date and location of vaccine, type of vaccine, lot number, expiry date, body part where the vaccine was injected, etc.

### Infection Information—(e.g., Covid-19 Information)

In some embodiments, an emergency profile includes health information relating to historical and/or ongoing epidemics or pandemics (e.g., Covid-19 information). The provisioning of emergency profiles accessible by emergency handlers or responders and/or public health personnel can be particularly helpful for response to and containment of infectious diseases, such as Covid-19. People who have Covid-19 symptoms may register and provide information for creating emergency profiles. In the event of an emergency (e.g., a 911 call), the emergency service provider can be provided with relevant emergency data about the user for the emergency response.

When responding to emergencies involving Covid-19 patients, emergency responders can be prepared with appropriate personal protective equipment and protocols to reduce their risk of infection during the emergency response. In the case of a person whose profile indicates they have recently tested positive for Covid-19, for example, an emergency responder may put on an N95 mask beforehand and subsequently isolate or quarantine the patient within the healthcare facility. In addition, the specific patient's health profile can include critical medical information that responders need to be aware of in order to provide effective medical care during the emergency response (e.g., allergies).

Emergency Data Sessions with User Devices

In further embodiments, the EMS establishes a first data channel with a user device and a second data channel between the EMS and the ESP, wherein the EMS bridges the first and second data channels to enable the communication device and the ESP to communicate. In some embodiments, the EMS converts data (e.g. data set) from the communication device into a format suitable for the EDC (e.g. analog or digital, audio, SMS, data, etc.) before sending or routing the formatted data to the EDC. In some embodiments, the EMS routes communications to a device associated with a first responder. In some embodiments, the communication device relays additional communications, information, and/or data sent or shared between member devices in the group of devices to the EMS or ESP after a request for assistance has been sent. In further embodiments, the additional information is relayed to the EMS or EDP after the request for assistance has been sent in order to provide current information that is relevant to the request. For example, in some instances, communications between member devices contain information relevant to the emergency (e.g. information that the user who is experiencing a medical emergency suffers from diabetes). Accordingly, in some embodiments, the information is sent autonomously, at request of a user of the communication device, or at request of the recipient (e.g. ERDP, ESP, emergency responders, etc.).

In some embodiments, the ERDP may contact (call or send SMS to) the user device, associated devices and/or emergency contacts. In some embodiments, the EMS will contact sequentially. For example, the ERDP will call the next number on the list if a user was not responsive in the previous contact number. The advantage of sequentially going through an emergency contact list is that the user can designate contacts of other closely associated users on the top of the list (e.g., close family, physicians, etc.). In the same way, the user may give lower priority to emergency contacts who would only be contacted in the event that other users are unavailable.

In some embodiments, a prioritized list of contacts comprises at least one associated device of the specific user. In some embodiments, a prioritized list comprises a plurality of associated devices having an order of priority. In further embodiments, the order of priority determines the sequence in which associated devices in the prioritized list are contacted by, for example, an emergency management system or a dispatch center. As an example, a prioritized list comprises a user's cell phone and tablet with the cell phone having a higher priority than the tablet (e.g. as determined by the user), wherein the cell phone and tablet are associated devices of a triggering device (e.g. a wearable heart rate monitor). The user has a heart condition and has adjusted the settings for the heart rate monitor to detect an emergency when it detects an irregular heartbeat. In this case, the heart rate monitor detects an irregular heart beat and transmits an emergency alert. An emergency management system receives the emergency alert and looks up a list of associated devices of the triggering device. In this case, the associated devices are the cell phone and tablet. The emergency management system then connects to the cell phone since it has the highest priority in the list and obtains location information and other user information (e.g. user identity, medical information).

In some cases, it may be necessary to confirm if the emergency contact is also facing an emergency (e.g., a car accident affecting several members of the family). Once a contact picks up the call, the EMS determines whether that user is the one who is in the emergency situation. To determine this, the user may be asked a simple question such as “Are you in an emergency?” or “Do you need help?” For a quick response, the EMS will prompt the contact to respond by pressing a button on a keypad or by yes or no voice answer. If the connected user is in the emergency, an abridged message may be played such as “Do you want to be connected to 911?” or “Press 1 to connect to get connected to emergency dispatch center in your area.” If the contact is also not the one in the emergency situation, the EMS will play a detailed message to explain the emergency situation. An example detailed message may say: “we have detected that John Miller's car may be in an emergency on 1-90. Emergency responders are on the scene and John Miller is being transported to hospital.”

In emergencies, it is preferred that the communications are clear and concise. The EMS may send notifications to the specific user and emergency contacts about the status of the emergency as described in the Examples. The specific user and emergency contacts may be prompted to validate their devices to get status updates. In some cases, responses may be needed in a specified manner (e.g., by pressing buttons and having the option to repeat the question).

### Emergency Contacts

FIG. 8C illustrates how a user can enter emergency contacts who may be authorized to receive notifications and status updates about the user during an emergency. As shown, emergency contacts may be relatives of the user. In other embodiments, emergency contacts may include physicians, employers, landlords, public health officials, etc.

The EMS may contact the emergency contact through various ways including automated emergency flows as shown in FIG. 2. The emergency contacts may be prompted to verify their devices before receiving notifications. Notifications may be in the form of calls, SMS, email or another form of electronic communication. In some embodiments, the notifications may be in the form of text-to-speech calls to devices associated with the emergency contacts.

### Medical Service Providers

In some embodiments, physicians (primary care or specialist) may be contacted after the emergency has been triggered. A user can provide a doctor's name and contact, which may be included in the EP. In some embodiments, the doctor may be informed when there is an emergency. For example, the doctor may be informed which hospital the user is being taken to during a medical emergency. In some embodiments, medical insurance information may also be saved in an EP, including in-network medical facilities and medical service providers. This information may be helpful for the user's treatment.

FIG. 8D illustrates how the emergency data in the emergency profile can be updated. As shown, different sections of the EP can be updated at different times. In some embodiments, the timestamp of the update will be included in the EP, so that the age of data is visible.

When an emergency is triggered (e.g., an emergency call, emergency data session, user-triggered panic button, sensor-triggered alert, etc.), the EMS may locate the user data in the emergency profile database using the phone number of the specific user. In some cases where sensors have triggered the emergency, the EMS may attempt to contact the user (e.g., a keyholder) at one or more devices to confirm the emergency.

After the emergency has been triggered, the EMS may retrieve information about one or more emergency contacts. The user may have provided a list of emergency contacts during the registration at the EP portal or in the emergency data from other data sources. The emergency contact information may include the name of an emergency contact, the relationship with the user and the emergency contact's phone number and/or other contact information (e.g., email).

When an emergency is triggered, the EMS may locate the emergency data in the emergency profile (EP) database. In some embodiments, federated queries to third-party databases are sent after the emergency is triggered for privacy and security reasons. The retrieved data is processed in various ways and provided to the ESP (e.g., display shown in FIG. 10).

### Federated Query of Multiple Sources

It is advantageous to obtain data from two or more sources to get comprehensive and reliable data. Even when a user enters data through a portal, there may be discrepancies and typographical errors. In addition, the user may not regularly update the information and the most recent data may not be entered. Thus, a federated query involving two or more sources may be desirable to get current and reliable information. In some cases, the same data parameter or field can be cross-referenced between multiple sources to improve accuracy.

A federated query refers to querying one or more databases and/or servers to gather relevant information to be displayed for emergency response. Thus, the federated query can refer to querying multiple databases and receiving several query responses. In some embodiments, the federated query is made simultaneously, while in other embodiments, sequentially. The retrieved data from the federated query is processed to obtain a federated response that consolidates information from the various individual queries.

Various modes for queries may be used. For example, some legacy systems require specific protocols and allow for specific identifiers for querying. Modern systems may use API endpoints for sending queries and various identifiers may be allowed.

In some embodiments, the EMS determines what type of query to send and which identifier to use. In one example, the user may not have provided a social security number, but a medical insurance database may require the query to include the SSN. The EMS may send a query to a first database using the user's name and DOB to obtain the SSN in the retrieved data. Using the identifier obtained in the retrieved data, the EMS may query the medical insurance database using the SSN to obtain information about in-network physicians and hospitals.

As another example, the user provides EP data that does not include blood type. However, as blood type information may be needed during a medical emergency, blood type information may be obtained by querying one or more third-party servers or databases (e.g., MedicAlert).

### Rules for Federated Query

Retrieved data from the federated query may be processed to generate EP data to be displayed at the ESP using one or more rules. The rules are aimed to increase reliability of the data, so that it can be used for emergency response. In some embodiments, the retrieved data from the federated query is used to populate the plurality of data fields to generate the emergency profile of the specific user in the EP database, which may be displayed at an appropriate ESP in case of an emergency.

The use of multiple sources in a federated query is beneficial, but there may be discrepancies and errors in the data, which can overwhelm and misdirect ESP personnel. Thus, the retrieved data may be processed before displaying at the ESP. In some embodiments, the retrieved data from the federated query is consolidated based on one or more rules for federated query.

Some non-limiting and illustrative examples of rules are listed below. Regarding rule (i), data that is more recent can be expected to be most accurate. Some sources may be known to be more reliable than others based on feedback or other scoring mechanisms. Finally, the data from two sources can be cross-validated. For example, if the blood-type is B positive in two sources, the reliability increases (see FIG. 10 where the two sources indicated the same blood type). It is contemplated that feedback from ESP personnel will be helpful in identifying relevance and reliability of the sources and specific preferences of the ESP agency can also be taken into account.

(i) select data that is more recent;

(ii) select data from a source with higher reliability; or

(iii) select data based on cross-validation from two or more sources.

In some embodiments, another rule that may be implemented is that retrieved data that is not recent is excluded and not displayed at the ESP. Depending on the type of data, recent data is less than 1 minute to 10 years old. In some embodiments, recent data is within the last year. In some embodiments, recent data is within the last 6 months. In some embodiments, recent data is within the last two weeks. In some embodiments, recent data is within the last 10 minutes. For example, static data that does not change such as name, age, sex, blood type can be considered to be recent as a rule or for a longer time as compared to dynamic data. Static data could also change over time such as home address and may may remain recent for a shorter time (e.g., 6 months, 1 year). For dynamic data such as location of the user, environmental or health sensor data, the data may need to be recent if is less than 1 min-24 hours, preferrably within the last 10 minutes.

The sources that are queried for the federated query can also be selected for relevance and reliability. In some embodiments, the sources for the federated query are selected based on one or more of location of emergency, type of emergency, identification information, user permissions, ESP authorization and preferences, reliability of the sources, etc. For example, if a user's DOB or social security number is not available, some sources may not be usable. If the emergency is a medical emergency, databases with medical information may be prioritized first. Querying additional sources may be provided as an option to the ESP personnel. If there is a vehicular emergency, license plate databases can be searched. Examples of emergency profiles with a non-limiting list of data fields are shown in Table 1.

TABLE 1
Exemplary Emergency Profiles

| | | | | Phone | | |
|---|---|---|---|---|---|---|
| Source | Time/Day | Name | DOB | No. | Location | Data |
| Cook | Nov. 30, 2017 | Jane | Jun. 5, 1958 | (111) | 345 Green Ave, | Covid-19 |
| County | 15:06:43 | Miller | | 222-3333 | Brooklyn, NY | J&J; |
| | | | | | | Aug. 4, 2021 |
| EP | Nov. 30, 2017 | John | Apr. 9, 1980 | (111) | 345 Green Ave, | Depression; |
| | 15:06:43 | Miller | | 222-3434 | Brooklyn, NY | Anxiety |
| MedicAlert | Nov. 30, 2017 | William | May 6, 1990 | (121) | 122 Forest St, | Parkinson's, |
| | 15:12:23 | Carter | | 444-5555 | Brooklyn, NY | susceptible |
| | | | | | | to falls |
| Google | Nov. 30, 2017 | Beck | Aug. 4, 1967 | (121) | 389 Broad St, | Alternate |
| | 15:16:48 | William | | 333-4444 | Brooklyn, NY | user number |
| Sec. of | Nov. 30, 2017 | Jessica | Sep. 5, 1987 | (111) | 122 Forest St, | License |
| State | 15:25:18 | Smith | | 212-1212 | Brooklyn, NY | Plate: |
| | | | | | | 56764C |

In some cases, the data retrieved in response to the federated query is processed using one or more algorithms to provide a formatted and/or standardized data set for display at the ESP. The analysis of the retrieved data can include natural language processing (NLP). In some embodiments, natural language processing is used to parse data records. In some cases, NLP is used for part-of-speech tagging words in a data record, for example, the doctor's notes section of a medical record. In some embodiments, the method further comprises natural language processing (NLP) for performing text summarization on the data record.

In some embodiments, natural language processing is performed to provide a formatted and/or standardized data set for determining relevancy. The data set may be assessed for relevancy using various methods such as, for example, one or more predefined rules. NLP algorithms deal with how to program computers to process and analyze large amounts of natural language data and may involve speech recognition, natural language understanding, and natural language generation. NLP allows human-computer interaction for automatic text summarization, sentiment analysis, topic extraction, named entity recognition, parts-of-speech tagging, relationship extraction, stemming, text mining, machine translation, and automated question answering.

Instead of hand-coding large sets of rules for language, NLP algorithms can rely on machine learning to automatically learn these rules by analyzing a set of examples, and making a statistical inference. In general, analyzing a large volume of good training data leads to more accurate NLP models and algorithms.

In some cases, social media information for the specific user may be included in the analysis. NLP can be well suited for analyzing vast amounts of social media data. For example, sentiment analysis can be powerful in evaluating social media posts and classifying the text as positive, negative or neutral. NLP can be used for evaluating various social media analytics like trending topics.

In some embodiments, the systems, methods, and media described herein use one or more algorithms analyzing and/or processing the retrieved data. In some embodiments, machine learning algorithms are used for training prediction models and/or making predictions such as predicting whether an electronic medical record or information in the record is relevant to an emergency. For example, an emergency may be labeled with an emergency type such as medical, vehicle accident, crime/police, or fire, for which different kinds of information may be relevant to first responders. In some embodiments, the algorithm predicts a degree of relevance to an emergency. Various algorithms can be used to generate models that are used to identify data or information that is relevant to an emergency. In some instances, machine learning methods are applied to the generation of such models.

In some embodiments, a machine learning algorithm uses a supervised learning approach. In supervised learning, the algorithm generates a function from labeled training data. Each training example is a pair consisting of an input object and a desired output value. In some embodiments, an optimal scenario allows for the algorithm to correctly determine the class labels for unseen instances. In some embodiments, a supervised learning algorithm requires the user to determine one or more control parameters. These parameters are optionally adjusted by optimizing performance on a subset, called a validation set, of the training set. After parameter adjustment and learning, the performance of the resulting function is optionally measured on a test set that is separate from the training set. Regression methods are commonly used in supervised learning. Accordingly, supervised learning allows for a model or classifier to be generated or trained with training data in which the expected output is known in advance such as when the relevance is known. For example, a training data set may be curated to provide for labeled data that can be used to train the model to generate a trained algorithm that can then generate predictions or labels of relevance for unlabeled data.

In some embodiments, a machine learning algorithm uses an unsupervised learning approach. In unsupervised learning, the algorithm generates a function to describe hidden structures from unlabeled data (e.g., a classification or categorization is not included in the observations). Since the examples given to the learner are unlabeled, there is no evaluation of the accuracy of the structure that is output by the relevant algorithm. Approaches to unsupervised learning include: clustering, anomaly detection, and neural networks.

In some embodiments, a machine learning algorithm learns in batches based on the training dataset and other inputs for that batch. In other embodiments, the machine learning algorithm performs on-line learning where the weights and error calculations are constantly updated.

In some embodiments, a machine learning algorithm is applied to new or updated emergency data to be re-trained to generate a new prediction model. In some embodiments, a machine learning algorithm or model is re-trained periodically. In some embodiments, a machine learning algorithm or model is re-trained non-periodically. In some embodiments, a machine learning algorithm or model is re-trained at least once a day, a week, a month, or a year or more. In some embodiments, a machine learning algorithm or model is re-trained at least once every 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or more.

In some instances, machine learning methods are applied to select, from a plurality of models generated, one or more particular models that are more applicable to certain attributes. In some embodiments, different models are generated depending on the distinct sets of attributes obtained for various communications.

In some embodiments, the classifier or trained algorithm of the present disclosure comprises a feature space defining a plurality of features. In various embodiments, each feature space comprise pieces of information or parameters indicative of relevance. For example, features may include certain keywords or phrases having known meaning (e.g., identified using NLP). In some cases, predefined rules are used to determine relevance, for example, diagnostic status (e.g., Covid status) and health parameters such as heart rate and body temperature may be relevant to medical emergencies, while location may always be considered relevant regardless of emergency type. In some embodiments, the accuracy of the classification or prediction is improved by combining two or more feature spaces in a classifier instead of using a single feature space.

In some embodiments, an algorithm utilizes a predictive model such as a neural network, a decision tree, a support vector machine, or other applicable model. Using the training data, an algorithm is able to form a classifier for generating a classification or prediction according to relevant features. The features selected for classification can be classified using a variety of viable methods. In some embodiments, the trained algorithm comprises a machine learning algorithm. In some embodiments, the machine learning algorithm is selected from at least one of a supervised, semi-supervised and unsupervised learning, such as, for example, a support vector machine (SVM), a Naïve Bayes classification, a random forest, an artificial neural network, a decision tree, a K-means, learning vector quantization (LVQ), regression algorithm (e.g., linear, logistic, multivariate), association rule learning, deep learning, dimensionality reduction and ensemble selection algorithms. In some embodiments, the machine learning algorithm is a support vector machine (SVM), a Naïve Bayes classification, a random forest, or an artificial neural network. Machine learning techniques include bagging procedures, boosting procedures, random forest algorithms, and combinations thereof.

In some embodiments, a machine learning algorithm such as a classifier is tested using data that was not used for training to evaluate its predictive ability. In some embodiments, the predictive ability of the classifier is evaluated using one or more metrics. These metrics include accuracy, specificity, sensitivity, positive predictive value, negative predictive value, which are determined for a classifier by testing it against a set of independent cases (e.g., communications). In some instances, an algorithm has an accuracy of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a specificity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a sensitivity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a positive predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances an algorithm has a negative predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein.

In some embodiments, the retrieved data undergoes natural language processing using one or more machine learning algorithms. In some embodiments, the one or more machine learning algorithms utilize word embeddings that map words or phrases to vectors of real numbers. In some embodiments, the mapping is generated by a neural network. In some embodiments, a machine learning algorithm is applied to parse the text obtained from the retrieved data (optical character recognition extracted text from a PDF medical record). In some embodiments, a machine learning algorithm is applied to segment words into morphemes and identify the class of the morphemes. In some embodiments, a machine learning algorithm is applied to identify and/or tag the part of speech for the words in the multimedia content (e.g., tagging a word as a noun, verb, adjective, or adverb). In some embodiments, a machine learning algorithm is applied to classify the data record or portions thereof into a category such as relevance (e.g., relevant or irrelevant to an emergency or degree of relevance).

FIGS. 9A & 9B depicts a method for implementing federated queries for emergency data.

In some embodiments, the retrieved data from the federal query is displayed without processing. In some embodiments, the retrieved data indicates the source of the data and time that it was stored. In some embodiments, the retrieved data indicates a reliability score, which indicates how trustworthy a source is and how accurate the data is likely to be. In some embodiments, the retrieved data indicates a relevancy score, which indicates how relevant a particular data is to the specific emergency. For example, medical conditions are particularly relevant during a medical emergency while behavioral conditions may be relevant in all types of emergencies.

In some embodiments, when the emergency data is stored at a third-party server and receives a request for emergency data from the ERDP 110, as a database query, the third-party server formats the requested emergency data and stores this information in an alternate database, and forwards either a response or a reference to the alternate database for accessing the emergency data requested by the ERDP 110, which is provided to the ESP 130 over a hybrid analog and/or a data communication channel, depending on the capabilities of ESP 130. In some embodiments, the third-party server stores the emergency data, requested by the ERDP 110 or directly by the ESP 130, in the alternate database for a certain period of time after receiving the request for the emergency data regarding a user and any electronic devices 100. In some embodiments, this period of time is a timer value (e.g., a timer countdown or a set time point) defined by the ERDP 110 and the third-party server in conjunction with each other prior to the addition of the requested emergency data to the alternate database at the third-party server. In some embodiments, once the timer value has passed and no new requests for the emergency data pertaining to the particular user and the electronic device 100, or other devices associated with the user, are received by the third-party server, then the third-party server marks the particular alternate database entries to be deleted and waits for another, different, time-out interval. In some embodiments, once this particular second time-out interval has also been completed and no new requests for location data for the particular user or associated electronic devices 100 are received by the third-party server, the third-party server removes the specific marked entries from the alternate database in the next cycle of updates for the alternate database. In some embodiments, after adding the emergency data in the alternate database by the third-party server, the third-party server keeps updating the emergency data in the alternate database on a periodic, or as-needed basis, for the purpose of keeping the emergency data about the user or electronic device 100 current for providing the most recent and accurate emergency data to the ERDP 110 and the ESP 130 for the purposes of responding to a request for emergency assistance. In some embodiments, the third-party server is updated by the ERDP 110 for all the emergency data pertaining to all users and their associated electronic devices 100 that are served by the ERDP 110 at any current time. In some cases, data is marked for deletion depending on one or more factors such as age of data (e.g., current or old/out-of-date information, medical or confidential information). As an example, medical information protected under privacy laws such as HIPAA may be flagged for automatic and/or periodic deletion. Therefore, in emergency situations, such information may be requested from third-party sources, assessed for relevance, displayed to the ESP during the course of the emergency, and then deleted once the emergency is concluded or some time thereafter.

FIGS. 10 & 11 depicts interfaces displayed at an ESP system. Specifically, FIG. 10 shows an interface for showing EP data about an emergency as an overlay on the ESP map display. The ESP user may have to select a phone number from the queue 1010 or enter a phone number in the search box to be able to view the EP, if available. For example, the EP for 984-562-4564 1012C is displayed. The location of the emergency 1024C is displayed in interactive map 1020 (selected emergency may be designated with a different representation. The geofence boundary 1026C for the ESP may also be displayed on the map. During a medical emergency, the EP can be a valuable resource for responders.

FIG. 11 depicts another illustrative example of an interface displayed at an ESP system. The user's EP indicates that she has schizophrenia, which can be critical data during the emergency response (interface may highlight critical data with fonts, colors and other design features). Here, the ESP user (e.g., a telecommunicator who has picked up the emergency call), could be provided a prompt to contact Marcus resources (e.g., mental health professional, peer recovery specialist).

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1—Covid-19 Protocol

Pat, an elderly man, lives in an assisted living apartment. He suffers from a medical condition that makes him susceptible to falls. Recently, Pat has been feeling some symptoms of Covid-19 and tells his son Joseph that he is worried that he may have to go to the hospital anytime. Due to social distancing guidelines, Joseph is unable to visit his father or take him to the hospital if needed. As a precaution, Joseph uses an online portal to register an emergency profile for his father, which includes an indication that he has a fever and dry cough.

When Pat realizes that his breathing has become impaired, he calls 911 in the middle of the night. The data that was saved in the EP is queried and sent to the appropriate PSAP serving the jurisdiction. The PSAP call taker, Mary answers the call and opens up the emergency response application display with the map showing Pat's location and the EP data. Mary notices that Pat is showing Covid-19 symptoms and makes a note for dispatch to implement Covid-19 protocols.

In addition, vaccination information for Pat can be gathered via a query to local county records, private pharmacy and hospital records using Pat's first and last name. The vaccination information indicates that Pat's Covid-19 vaccine was more than 6 months ago and his immunity to Covid-19 may be impacted.

When EMTs arrive on the scene to take Pat to an in-network hospital, they are in full PPE and have provisions for providing oxygen on route. Pat makes a quick recovery after a few days in the hospital.

### Example 2—Covid-19 Status Updates to Emergency Contacts and Others

In most cases, family members are not allowed to accompany patients when they call an ambulance. Also, family members of individuals who may have COVID19 may not live with or near that individual. Even though family members may be aware of that individual's current health status, a situation may arise wherein that individual becomes suddenly short of breath or experiences a sudden increase in fever (usually happens at night). As a result, the individual may dial 9-1-1 without notifying any concerned family members. The family members may not be allowed to visit the hospital, but would like to get status updates.

In the field where the individual inputs Emergency Contact Information, a checkbox may be present under each emergency contact providing authorization for the emergency contact to receive status updates, e.g., the individual has dialed 9-1-1, an ambulance has been dispatched, the ambulance is taking the individual to Mercy Hospital where the emergency contact can call 212-999-9999 to follow up.

Once the citizen profile is submitted by the individual, all emergency contacts that have been authorized to receive status updates will receive a request to confirm their identity and agree to receive the updates. If the emergency contact input by the individual responds that they have been misidentified, a request to update that emergency contact will be sent to the individual.

### Example 3—Covid-19 Symptoms and Follow-Up Protocols

Some individuals may be experiencing symptoms of varying degrees but are directed to stay at home and monitor themselves. These individuals could be encouraged to sign-up on the citizen data website and list current symptoms. Responders may require information pertaining to an individual requesting emergency services, particularly if that individual is ill and presenting with certain symptoms.

When inputting information into the profile, in the ‘My Medical Information’ section, there can be a field to enter current symptoms or even have a list of symptoms and allow the individual to check off each symptom by tapping a box next to the symptom (e.g., maybe there can be more functionality such that, for instance, if the user checks the box next to ‘Fever’ then an input field can appear where the individual can input the temperature).

The system may automatically reach out to the individual for updated symptom information if certain conditions are met (certain symptoms are checked off). Additionally, the system may ask if that individual has been to a hospital in the last seven days (or some other pertinent time period) and ask for input as to what happened during that visit either through an input field or with checkboxes (e.g., received fluids, was there for 3 hours, etc.)

### Example 4—Behavioral Health Dispatch

A user, John Miller, suffers from severe depression and anxiety (see Table 1). His sister, Mary was worried about John being on his own. She had filled out his emergency health profile indicating that he suffers from mental health conditions. When a 911 call comes in from a neighbour about an altercation at John Miller's home, the call taker notes the mental health conditions. The dispatcher sends a behavioral health professional with a police officer to check on John. When they arrive, John is in an agitated state due to his fear that his neighbour is not wearing a mask and may have Covid-19. The responders are able to calm him down and de-escalate the situation.

### Example 5—Federated Query

An emergency call comes in for a medical emergency for Jane Miller in Chapel Hill (984-562-4564) (see FIG. 10). Querying EP database indicates that her blood type is “B positive” while another database indicates that her blood type is “B negative.” Based on the higher reliability of the EP database with user-entered data over the second database, the blood type is indicated as “B positive” in the ESP display. The source and age of the data will be displayed. When a third source, Medic Alert also lists the blood type as “B positive”, the data is cross-validated and a validation indicator is also shown.

What is claimed is:

1. A method for providing an emergency profile for emergency response, comprising:
collecting emergency data comprising identifying information for a specific user from two or more sources;
generating an emergency profile comprising a plurality of data fields populated with the emergency data for the specific user;
storing the user profile on a database comprising a plurality of emergency profiles for a plurality of users;
receiving an emergency alert regarding the specific user;
querying the database to locate the emergency profile comprising the plurality of data fields populated with the emergency data for the specific user obtained from two or more sources; and
transmitting at least a portion of the emergency profile of the specific user to an appropriate emergency service provider (ESP).

2. The method of claim 1, wherein at least a portion of the plurality of data fields is provided to an ESP device for display, wherein at least one of the two or more sources of the emergency data is indicated.

3. The method of claim 1, wherein collecting emergency data comprises providing a portal for entering emergency data regarding the specific user.

4. The method of claim 1, wherein at least a portion of the plurality of data fields for one or more emergency profiles is populated by references to two or more databases.

5. The method of claim 1, wherein the emergency data comprises retrieved data obtained from the two or more sources using a federated query.

6. The method of claim 5, wherein the retrieved data from the federated query is used to populate the plurality of data fields to generate the emergency profile of the specific user.

7. The method of claim 5, wherein the retrieved data from the federated query for a data field is consolidated based on one or more rules for federated query.

8. The method of claim 7, wherein the one or more rules comprise at least one of:
select data that is recent;
select data from a source with higher reliability; or
select data based on cross-validation from two or more sources.

9. The method of claim 5, wherein the two or more sources for the federated query are selected based on one or more of location of emergency, type of emergency, identification information, user permissions, ESP authorization and preferences, reliability of the two or more sources.

10. The method of claim 1, wherein the emergency profile comprises symptoms, test results, pre-existing conditions, allergies, medications, prescriptions, vaccination status, physician information, medical insurance, in-network medical facilities, emergency contact information, blood type, mental health conditions, substance abuse disorder, developmental disorders, or any combination thereof.

11. The method of claim 10, wherein collecting the emergency data for the specific user comprises providing an online portal allowing the specific user to enter at least a portion of the emergency data and optionally upload one or more files, photos, or videos.

12. The method of claim 1, further comprising flagging the emergency profile of the specific user based on an indication of a mental health condition and providing the ESP with additional options to dispatch mental health professionals in the vicinity of the emergency.

13. The method of claim 1, further comprising notifying one or more emergency contacts in the emergency profile of the emergency.

14. A system comprising:
a) a database comprising a plurality of emergency profiles for a plurality of users; and
b) at least one server providing an emergency management system server application, wherein the application is operative to:
collect emergency data comprising identifying information for a specific user from two or more sources;
generate an emergency profile comprising a plurality of data fields populated with the emergency data for the specific user;
store the user profile on the database comprising the plurality of emergency profiles for the plurality of users;
receive an emergency alert regarding the specific user;
query the database to locate the emergency profile comprising the plurality of data fields populated with the emergency data for the specific user obtained from two or more sources; and
transmit at least a portion of the emergency profile of the specific user to an appropriate emergency service provider (ESP).

15. The system of claim 14, wherein the application is operative to provide at least a portion of the plurality of data fields to an ESP device for display, wherein at least one of the two or more sources of the emergency data is indicated.

16. The system of claim 14, wherein the emergency data comprises retrieved data obtained from the two or more sources using a federated query.

17. The system of claim 16, wherein the retrieved data from the federated query is used to populate the plurality of data fields to generate the emergency profile of the specific user.

18. The system of claim 16, wherein the retrieved data from the federated query for a data field is consolidated based on one or more rules for federated query.

19. The system of claim 18, wherein the one or more rules comprise at least one of:
select data that is recent;
select data from a source with higher reliability; or
select data based on cross-validation from two or more sources.

20. The system of claim 16, wherein the two or more sources for the federated query are selected based on one or more of location of emergency, type of emergency, identification information, user permissions, ESP authorization and preferences, reliability of the two or more sources.
